(19) United States
(12) Patent Application Publication
(10) Pub. No.: US 2008/0221666 A1
(43) Pub. Date: Sep. 11, 2008

(54) STENT SYSTEMS

(75) Inventors: David Licata, Marina del Rey, CA (US); Sudip Pandya, Sunnyvale, CA (US); William R. George, Santa Cruz, CA (US); Roman Turovskiy, San Francisco, CA (US); Dai T. Ton, Milpitas, CA (US); James C. Liu, Sannyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CardioMind, Inc., Sunnyvale, CA (US)

(21) Appl. No.: 11/957,211

(22) Filed: Dec. 14, 2007

Related U.S. Application Data
(60) Provisional application No. 60/870,169, filed on Dec. 15, 2006.

(51) Int. Cl. A61F 2/06

(52) U.S. Cl. 623/1.22; 623/1.11; 623/1.15

(57) ABSTRACT

Medical devices and methods for delivery or implantation of prostheses within hollow body organs and vessels or other luminal anatomy are disclosed. The subject technologies can be used in the treatment of atherosclerosis in stenting procedures or be used in variety of other procedures. The systems can employ a self expanding stent restrained by one or more members released by an electrolytically erodable latch.

### CROSS-REFERENCES TO RELATED APPLICATIONS

This application claims the benefit of U.S. provisional application Ser. No. 60/870,169 (Attorney Docket No. 022037-001100US), entitled, “Stent Systems,” filed Dec. 15, 2006, which is incorporated by reference herein in its entirety.

### BACKGROUND OF THE INVENTION

Implants such as stents and occlusive coils have been used in patients for a wide variety of reasons. One of the most common “stenting” procedures is carried out in connection with the treatment of atherosclerosis, a disease which results in a narrowing and stenosis of body lumens, such as the coronary arteries. At the site of the narrowing (i.e., the site of a lesion) a balloon is typically dilated in an angioplasty procedure to open the vessel. A stent is emplaced within the lumen in order to help maintain an open passageway. Restenosis can be avoided by means of the scaffolding support of the stent alone or by virtue of the presence of one or more drugs carried by the stent.

Various stent designs have been developed and used clinically, but superelastic self-expandable, and balloon-expandable stent systems are predominant. Described herein are unique devices, systems and methods for self-expanding stent delivery and other applications.

### BRIEF SUMMARY OF THE INVENTION

The devices, systems and methods described herein are done so by way of exemplary embodiments. These embodiments are discrete examples only and in no way should be interpreted as limiting the inventions. The devices, systems and methods described herein address holding a radially-expandable implantable prosthesis (such as a stent) that is twisted down into a compressed or collapsed configuration for delivery. Such systems are detailed in U.S. patent application Ser. No. 11/265,999, which published under U.S. Patent Application Publication No. U.S. 2007/0100414 on May 3, 2007, and U.S. patent application Ser. No. 11/266,587, which published under U.S. Patent Application Publication No. 2006/0111771 on May 25, 2006, the disclosure of each these applications and publications being hereby incorporated herein by reference in its entirety. In the above-referenced systems, a cage or lattice type stent is held in a twisted (and therefore compressed) configuration through interface with one or more tabs, extensions or projection features at its end(s).

According to one embodiment of the invention, a stent delivery system comprises a delivery guide body having a distal portion and at least one elongate member including an elecrolytically erodable section; a stent comprising a near end, a far end and a structure extending therebetween; at least one of the near and far end of the stent held in contact with the elongate body; wherein release of the erodable section initiates stent release, and wherein an intermediate polymeric covering member is interposed between the erodable section and the seat.

According to another embodiment of the invention, a stent delivery system including an elongate delivery guide comprises a stent comprising a near end, a far end and a structure extending therebetween, the stent further comprising a near and a far mating portion at the near and far ends of the stent, near and far seats at a far portion of the delivery guide, a mating portion being received in each seat, at least one helical wrap including an electrolytically erodable section, the wrap at least partially covering at least one of the seats and mating portions received therein, and an insulative polymer sleeve interposed between the wrap and the mating portions.

According to another embodiment of the invention, a stent delivery system including an elongate delivery guide comprises a stent comprising a near end, a far end and a structure extending therebetween, the stent further comprising a near and a far mating portion at the near and far ends of the stent, near and far seats at a far portion of the delivery guide, one mating portion being received in one seat and the other mating portion being receiving in the other seat, one of the seats being rotatable, and near and far restraints for holding portions of the stent in a compressed state, one of the restraints including a helical wrap having an electrolytically erodable section, the wrap at least partially covering one of the seats and mating portions received therein.

According to another embodiment of the invention, a method of loading a stent delivery system comprises securing the first end of a stent having first and second ends to a first seat that is fixed to a delivery guide, the first end being secured to the first seat with a wrapping member, securing the second end of the stent to a second seat that is coupled to the delivery guide, twisting the stent into a twisted configuration while the second end of the stent is secured to a second seat with a restraint, and fixing the second seat to the delivery guide.

According to another embodiment of the invention, s method of implant delivery comprises introducing an implant delivery system in an electrolytic fluid; and applying electrical power to a delivery guide having at least one electrolytically erodable member, the power having an AC voltage component with a peak-to-peak configuration of at least about 5V, and a DC voltage signal of at least about 1V, wherein the DC component is increased from zero to a maximum over a period of at least about 0.1 seconds. In another embodiment, the DC component is increased from zero to a maximum over a period of at least about 0.5 seconds.

According to another embodiment, an implant delivery guide body comprises an elongate body, the body comprising a proximal metal tube, a distal metal tube, a corewire, and a superelastic helical wrap, the core wire connecting the proximal and distal tubes, the wrap overlaying at least one junction between the proximal and distal tubes.

According to another embodiment, a stent delivery system comprises an implant delivery guide body comprising a proximal metal tube, a distal metal tube, a corewire, and a superelastic helical wrap, the core wire connecting the proximal and distal tubes, the wrap overlaying at least one junction between the proximal and distal tubes, and a stent releasably mounted adjacent a distal end of the guide body.

According to another embodiment of the invention, a stent delivery system comprises an elongate delivery guide body, and a stent releasably secured to the delivery guide body, the stent held in a twisted, compressed profile for delivery, over a mandrel, a plurality of hollow cylindrical members interposed between the stent and the mandrel, wherein the hollow members are rotatable about the mandrel at least prior to holding the stent in its delivery profile.

According to another embodiment of the invention, a method of loading a stent delivery system comprises rotating at least one of a stent over a mandrel, onto a plurality of rollers on the mandrel; progressively spinning the rollers as the stent progressively assumes a compressed diameter, and securing the stent to the delivery system.

According to another embodiment of the invention, a self-expanding stent comprises a body portion have a closed cell lattice construction, a longitudinal axis when in relaxed state, and distal and proximal ends; a plurality of distal projections extending from the distal end and a plurality of proximal projections extending from the proximal end, the distal and proximal projections extending in a direction generally parallel to the longitudinal axis, and the proximal projections being longer than the distal projections.

According to another embodiment of the invention, a stent delivery guide comprises a delivery guide having a first length having a proximal and distal end portion, a second length having a proximal and distal end portion, a self-expanding stent having a proximal and distal end portion, and a coil having a proximal and distal end, the first length distal end portion being coupled to the second length proximal end portion, the second length distal end portion being coupled to the proximal end of the stent and the distal end of the stent being coupled to the proximal end of the coil, which forms the distal tip of the delivery guide, the first length being less flexible than the second length.

According to another embodiment, a self-expanding stent can comprise a tubular nitinol alloy body having two open ends and including a plurality of interconnected struts meeting at junctions, and a plurality of projections located at adjoining struts at each end, the projections having a centerline offset from a centerline of an adjacent strut junction.

In one embodiment, the tabs/extensions/projections are adapted to nest or otherwise interface with complimentary seat features set upon or retained by the guide body portion of the implant delivery system. The projections can include elongate members that offer a laterally stable interface or can include hook-shaped forms (e.g., “J”, “T”, “L”, “S”, “V”, shapes and the like) that also provide an axially stable interface. A grasping form of interface can be employed to axially tension the implant and/or provide secure capture at one side of the implant (e.g., to provide “bail-out” or retrieval potential from partial deployment).

The delivery guide side of the interface can be referred to as a “seat” or otherwise. Especially, where the members hook into one another, they can be regarded as “nested” or “nesting” features. “Interlocking” or “lock-and-key” terminology can also be used to describe the interface features.

In another embodiment, the implant/delivery guide interface can be adapted for sliding receipt and release. Such configurations enable various self release or automatic release approaches. For these types of interfaces, “key” and “way” terminology can be most appropriate. Still, the delivery device can be regarded as carrying a “seat” or “seating” region or portion.

The implant can have symmetrical ends. In other words, the implant can utilize the same type of projection on each side. In other variations, differently configured ends can be used. In either case, the delivery system seat features are typically coordinated in their configuration.

Even if the ends are “symmetrical” as described above, in one exemplary embodiment, the tabs are offset from a centerline of an adjoining cell. Here, all the tabs are advantageously offset in a coordinated fashion such that when the body of the stent is twisted, the leverage applied to adjacent strut junction or “crown” members causes them to more closely conform to the delivery guide outer diameter.

Considered otherwise, the offset location of the tab(s) connection to adjoining strut junctions or crown features can provide a laterally-displaced point of rotation around which the crown(s) rotate until they lie substantially flat on the delivery guide. By way of comparison, when the tab pivot location is centrally disposed, shorter lengths on either side more easily tilt at an angle relative to an ideal flat packing since the interference is relatively less. Larger (substantially) flat panel sections of the tabs (of similar width to the delivery guide body) contact the delivery guide body to limit further rotation and offer improved packing geometry for the loaded stent.

In this embodiment, the stent can comprise a tubular NiTi alloy body having two open ends and including a plurality of interconnected struts meeting at junctions, with a plurality of projections located at adjoining struts at each end, where the projections are oriented along a centerline offset from a centerline of an adjacent strut junction. In use, the projections are offset from a centerline of the strut junctions in a direction opposite the intended direction of twist for the stent.

Typically, the struts define a fully closed-cell stent design. As such, the full diameter of the stent can be reduced without need for additional restraining means to hold down unconstrained sections. Further, a reduced cross-section region can be provided between each strut junction and each projection. The maximum degree of offset is typically limited by interference between adjacent projections when the stent is in a compressed configuration. As such, the potential offset is related to a number of factors including strut and projection width, adjacent crown configuration, and the like.

The projections in this exemplary embodiment are typically straight. Likewise, the projections are typically shorter than the struts that define the body of the stent (or other implant to be delivered). At as short as about 0.020 to about 0.010 inches the projections can provide a stable interface to releasably secure the implant to the delivery guide.

Implants equipped with projections as described herein can be releasably secured to the delivery guide in any of the exemplary embodiments described herein, in the filings incorporated by reference, or otherwise. A non-exhaustive list includes releasable members overlaying the projections selected from circular band(s), helical wrap(s) or one or more elongate sleeves.

Still, in combination with other features described herein, the stent tab or projection features can simply be configured to lie along a centerline of each adjacent cell as presented in the incorporated '999 and '587 patent applications. Another improvement to the stent concerns the geometry of the cell pattern. Specifically, an improved packing cell pattern can be used, such as that described in U.S. patent application Ser. No. 11/238,646, which is incorporated by reference herein in its entirety.

In the pertinent approach described therein, a final or near-final stent cut pattern is generated by expansion (performed physically or by computational methods) from an idealized packing geometry. An improvement to that approach contemplates accounting for a twist or helical element in the intended packing geometry.

Specifically, exemplary methods of design and/or manufacture are contemplated in which a precursor stent strut design is first provided in a desired compressed configuration, the precursor stent having struts aligned in a helical orientation for optimized packing. The precursor stent design (as a physical stent or single element by computational methods) can then be expanded and untwisted to a desired expanded configuration. This expanded configuration can fully untwist the stent, or a helical arrangement of the expanded cells can persist.

The expanded pattern can then be used to set a stent cutting pattern. The pattern can correspond exactly or it can serve as a generic template. In one exemplary embodiment, the projection of a photograph of the strut pattern is projected or copied onto a cylindrical body, which is then followed approximately in setting final cut geometry to produce the stent from tubing (e.g., superelastic NiTi tubing).

In a preferred exemplary embodiment, the precursor stent design is provided at a fully compressed diameter with the degree of desired twist. However, it can be provided at as much as about 50% of the fully compressed diameter. The number of turns or degree of twist in which case is preferably kept to as many turns as required to fully compress the stent to its minimum diameter. This can ensure more accurate reversibility of the process. However, some lesser number of turns or twists can be acceptable, given the performance of the resultant design and the already exemplary performance of stents generated according to the '646 method, improved upon with the twist element described herein.

These various embodiments can yield a self-expanding stent having a plurality of struts, with the stent having an expanded shape and a compressed shape turned, wrapped, or helically twisted about a central body. In the compressed, twisted shape, the struts can define a plurality of teardrop-shaped openings along substantially a whole length of the struts. Preferably, the teardrop shapes are formed over substantially an entire length of the struts and contact along the strut lengths can be avoided. However, by accounting for the additional stresses introduced by twisting, the reliability of obtaining the desired compaction pattern is improved with the improved processes described herein. The final product operates more independently of inconsistencies introduced in twist-loading the stents.

With any such stent configuration (or still others), the delivery guide can advantageously include a configuration in which at least one latch member has a wire or ribbon wrapped over or around the tab/projection features of the stent.

One improvement in the devices, systems and methods described herein over other related approaches described in the commonly-assigned above-referenced '999 patent application concerns an intermediate polymeric layer provided between the wrap wire and the seat that receives the stent ends. This layer serves a number of purposes. For instance, it offers an improved insulation barrier between metallic parts. It also provides a more uniform interface against the stent. The polymeric layer can comprise a high-strength polymer such as polyimide or PET (especially highly structure PET) to resist cutting or other damage during assembly. Alternately, a more lubricious polymer (e.g., PTFE) can be employed to assist with stent slide-out and release upon latch erosion.

Especially where a relatively lubricious polymer (or an inner layer of such a polymer) is provided in a multi-layer construction, the sleeve or wrap forming the layer (or layers) of the sleeve can be imperforate. When friction might be a concern for release, the sleeve can be cut open or scored or perforated to open along one or more sections. These can be configured as straight-line sections or otherwise. Two, three, four or more such “flaps” can be provided.

The sections can be adapted to open radially to allow for stent expansion as the wrapping member is released. The sections can generally be configured to open or otherwise flare outwardly along a significant length of the captured stent end projections. To minimize delivery system diameter (while accounting for the additional layer or layers), the polymeric sleeve (simple or composite) is preferably less than about 0.002 to about 0.001 inches thick, although greater thicknesses can be used. It can be as thin as about 0.0005 inches and still be sufficiently robust to operate as desired.

In one another exemplary embodiment of the delivery system, a wrap-style latch is provided only at one end of the stent carried by the delivery guide. At the opposite side of the stent, a latch system is provided such that untwisting of the stent causes the stent to shorten and draw the stent tabs/projections out of their interfacing seat region. The latter (untwist-style) latch will typically be released first to deliver the implant. A slidable band over the stent ends can be provided so that partial expansion of the stent during untwisting assists in complete release by driving the band off the stent tabs or projections.

In another exemplary embodiment, two wrap-style latches can be provided (per stent on a given delivery system). In this case, one of the latches to be released can be configured such that it can rotate with its underlying seat feature and the optional interposed polymeric layer during delivery device loading/construction. Once the stent is loaded onto the delivery system, however, the wrap/seat assembly is preferably secured against rotation.

The ability of the assembly to rotate during assembly addresses handling considerations of the wrap. By wrapping the second (be it near or far) wrap with the stent, assembly problems are avoided given that the wrap is secured at an inner portion or section of the corresponding projections. In other words, because the elements can be moved in unison, the wrap member (typically a fine wire running all the way to the proximal end of the delivery guide) does not have to be attached after wrapping or handled extensively in threading it opposite the direction of seat rotation. Such handling can damage a wire or, more particularly, the insulation or coating required to focus the point of electrolytic erosion for release.

To achieve a system in which the wire for the wrap is able to rotate with the stent-mating seat for capturing stent projections, the wire can be secured to the seat in one fashion or another. In one exemplary embodiment, the wrapping latch wire can be attached to a leg or finger extension of the seat. In another exemplary embodiment, a slot can be employed at an end opposite the stent mating portion of the seat in which the wrap wire is received. In the former embodiment, the wire is preferably attached to the extension over an intermediate insulative, polymeric sleeve over the projection. In the latter embodiment, an insulative polymer sleeve is first bonded to the wire end and this complex then bonded into the slot. In use, the wire underlies the seat body, making a turn toward the outside of the device.

However the members are secured (e.g., by epoxy, solder, laser welding, etc.), the wrap and sleeve can be configured to preferably rotate together during stent loading without damage to the components involved. Bonding approaches can generally be preferred to maintain electrical isolation between the seat and latch wire.

In another embodiment, once the stent is loaded with the wire wrapped over the stent tabs/projections, an end or medial section of the wire (not shown) can be wrapped over a fixed portion of the delivery guide and secured/bonded thereto. The seat can also be fixed or it can be allowed to float, with its position secured prior to wrap release by the wrap alone.

Use of the rotatable wrap/key assembly approach for loading a second side of a twisted stent can be practiced according to the method as described in the '999 filing, where one side of a stent is secured to a first seat fixed to a delivery guide with a first wrapping member over a first mating portion, the stent is twisted into a second configuration while an opposite side of the stent is secured to a second seat with a second wrapping member over a second mating portion, and the second seat and the second wrapping member are fixed to the delivery guide after rotation with the stent.

Naturally, the subject methods can include each of the mechanical activities associated with implant loading and system manufacture as well as navigation to a site and implant release. These methods can include those associated with angioplasty, bridging an aneurysm, or deploying radially-expandable anchors for pacing leads or an embolic filter. These methods can also include placement of a prosthesis within neurovasculature, within an organ selected from the kidney and liver, within reproductive anatomy such as selected vasdeferens and fallopian tubes. Still other applications can also be practiced. The lattice or cage-like stent structures can be adapted for various uses.

Regarding delivery system loading, additional device features can refine the method. In one exemplary embodiment, a plurality of rollers can be provided over the member around which the stent is wound. These members can be configured to roll or rotate, thereby incrementally supporting the stent as the members twist with it, for instance, twisted in a manner similar to the loading method described in the '999 application. This can improve the stent packing profile as compared to when the stent is twisted or wound around a solid mandrel.

Another exemplary embodiment improves overall system profile without significant negative impact on intended performance by the use of a transition coil. This transition coil spans a gap between first and second hypotube bodies connected by an underlying corewire—all of which serve as structural members for push/pull transmission and torque transmission of the delivery guide. Often, the transition coil will be carried forward of a junction (such as a solder joint) between the corewire and the distal hypotube. Here, the use of a NiTi coil (in the form of round wire or ribbon) is especially advantageous for small-scale systems where the junction outside diameter is larger than the relaxed diameter of the coil.

In one exemplary manufacturing approach, the NiTi coil can be rolled over and past the junction from a section already fed over the core wire and/or one hypotube section. At the point of wrapping and unwrapping, the NiTi alloy can be deformed to return to shape (either by superelastic or shape-memory behavior if heated).

Preferably, the transition coil/wrap is such that it does not substantially contribute to torque transmission characteristics. Otherwise, it could introduce uneven torque characteristics in clockwise vs. counterclockwise rotation due to compaction or unraveling of the coil. In any case, the transition coil advantageously offers a substantially uniform outside diameter to the delivery guide and protects underlying components. In certain embodiments of the delivery system, these components can include a pair of fine wires running parallel to the structural corewire used for primary torque transition to the distal hypotube. Typically, the outside diameter delivery guide will range from less than about 0.001 to about 0.002 inches and have a 0.014 crossing profile (i.e., be compatible for use with 14-thousands type catheters), although other dimensional configurations can be used.

In other exemplary embodiments, the system can use electrical actuation of the erodable member to release the implant. Methods as described in the '999 filing are contemplated and improved upon by “ramping” up and/or down the DC signal components. In one embodiment, the method of actuating the electrolytic latches (or joints in other systems such as well-known GDC devices) includes introducing an implant delivery system into an electrolytic fluid (e.g., the blood in a patient's vasculature) and applying electrical power to at least one electolitically erodable member of the delivery system having, where the power has an AC voltage component with a peak-to-peak configuration of at least about 5 volts (V), and a DC voltage component of at least about 1V. Such an approach allows for fast (less than several seconds) electrolytic erosion release times at low (and relatively safe) DC voltages. Further improvements to safety can also be realized in an exemplary method where the DC component is increased from zero to a maximum over at least about 0.5 seconds. The DC component can instead be ramped up over a period of about 1 second or more. Conversely, if it is desirable to conserve actuation time, the DC component can be ramped up and/or ramped down in between about 0.1 and 0.25 seconds. It should be noted, however, that any DC ramping time can be used in accordance with the needs of the application.

It can be desirable to configure the power supply device hardware and/or software so that the DC voltage component varies to deliver a constant current during electrolytic erosion. In one exemplary embodiment, the DC component can vary between about 1V and about 10V. In this or another embodiment, the AC voltage component can have a peak-to-peak configuration of 15V or less. Generally, a maximum effect from the AC component (described further below) is achieved with a substantially square-wave profile.

Whatever the combination of voltages, it will typically be the case that the power applied to the delivery guide includes (sometimes or always) a negative voltage signal at the members that erode. Moreover, the power applied to the delivery guide will generally be such that the power actually delivered to the erodable regions has an AC voltage component with a peak-to-peak configuration of at least about 5V, and a DC voltage component of at least about 1V.

Of the various features described, the delivery systems herein offer a number of advantages in their construction and ability to deliver implants with or without coatings for lubricity and/or drug delivery in various applications. Other systems, methods, features and advantages will be or will become apparent to one with skill in the art upon examination of the following figures and detailed description. It is intended that all such additional systems, methods, features and advantages be included within this description, be within the scope of the devices, systems and methods described herein, and be protected by the accompanying claims.

### BRIEF DESCRIPTION OF THE DRAWINGS

FIG. 1 is a perspective view of a heart showing an embodiment according to the invention (device 22) being delivered to a coronary artery and where a guide catheter GC can be used to deliver device 22 and with optional balloon catheter BC, which can be used for pre-dilation of the lesion to be treated and/or post dilation of the lesion after the stent has been placed.

FIG. 2A is a perspective view depicting an exemplary embodiment of an implant within a vessel.

FIG. 2B is an axial cross-sectional view depicting another exemplary embodiment of an implant within a vessel.

FIG. 3A is a perspective view depicting an exemplary embodiment of the delivery system.

FIG. 3B is a perspective view of a portion of the exemplary embodiment of FIG. 3A.

FIG. 3C illustrates packaging for elements of the embodiment of FIG. 1

FIG. 3D is a schematic view depicting an exemplary embodiment of the electrical hardware of FIG. 3A.

FIGS. 4A and 4B are schematic views depicting an additional exemplary embodiment of the delivery system.

FIGS. 5A and 5B illustrate proximal and distal portions of the embodiment illustrated in FIGS. 4A and 4B.

FIG. 6A diagrammatically illustrates a distal portion of the embodiment in FIG. 4A in a stent loaded state.

FIG. 6B diagrammatically illustrates the distal portion of FIG. 6A in a released state where the distal end of the stent is released.

FIG. 6C diagrammatically illustrates another distal portion embodiment according to the invention in a stent loaded state.

FIG. 6D diagrammatically illustrates a section of the portion shown in FIG. 6C with the stent tab restraint removed to show the stent tabs seated in a portion of the release mechanism.

FIG. 6E diagrammatically illustrates the distal portion of FIG. 6C in a released state where the distal end of the stent is released.

FIG. 6F is sectional view taken along line 6F-6F in FIG. 6C.

FIG. 6G is a longitudinal sectional view of a portion of the device shown in FIG. 6C.

FIG. 6H is a longitudinal section view of another portion of the device shown in FIG. 6C and illustrating one distal tip embodiment.

FIG. 6I illustrates a connector tube to connect a portion of the delivery guide distal section of FIG. 6H to the distal tip coil.

FIG. 6HI is a sectional view taken along line 6H1-6H1 in FIG. 6H showing the double flat corewire that extends from the distal tip coil.

FIG. 6J is a longitudinal section view of another embodiment of the delivery guide distal section showing a proximal stent release mechanism according to another embodiment of the invention.

FIG. 6K1 and 6K2 are perspective views of the release mechanism of FIG. 6J where FIG. 6K1 shows the stent tabs covered with an insulative sleeve and FIG. 6K2 shows the stent tabs uncovered.

FIG. 6K1a is a perspective view of the insulative sleeve of FIG. 6K1.

FIG. 6K2a is a perspective view of the stent tab seat of FIG. 6K2.

FIG. 6K3 is a sectional view taken along line 6K3-6K3 in FIG. 6J.

FIG. 6L1 and 6L2 are perspective views of a another release mechanism embodiment that can be incorporated into the embodiment of FIG. 6J where FIG. 6L1 shows the stent tabs covered with an insulative sleeve and FIG. 6L2 shows the stent tabs uncovered.

FIG. 6L2a is a perspective view of the stent tab seat of FIG. 6L2.

FIG. 6L3 is a sectional view of taken along a similar section as 6K3 in the embodiment with a wire finger as shown in FIG. 6L2.

FIGS. 7A-7F illustrate one method for loading a stent in the distal section of the delivery guide according to the invention.

FIGS. 7G, 7H, 7I, 7J, 7J1, 7K, 7L, and 7M illustrate another method for loading a stent in the distal section of the delivery guide according to the invention.

FIGS. 8A-B are schematic views depicting exemplary embodiments of the attachment of the proximal wrap 54 to the proximal seat 62.

FIGS. 10A-B are schematic views depicting additional exemplary embodiments of the delivery system.

FIG. 10C is a schematic view of region 10C of FIG. 10B.

FIG. 10D is a schematic view of region 10D of FIG. 10B.

FIG. 10E is a cross-sectional view of FIG. 10D taken along line 10E-10E.

FIG. 10F is a schematic view of region 10F of FIG. 10B.

FIG. 10G is a cross-sectional view of FIG. 10F taken along line 10G-10G.

FIGS. 10H-10J illustrate another embodiment of what is shown in FIGS. 10A-B; where FIG. 10H is longitudinal sectional view of a portion of the distal section of the distal guide proximal to the portion shown in 6J, FIG. 10I is a portion proximal to the portion shown in FIG. 10H and FIG. 10J is a sectional view taken along line 10J-10J in FIG. 10I.

FIG. 10K is a variation of the transverse section of FIG. 10J for the proximal portion of FIG. 10T.

FIG. 11A illustrates one embodiment of a power connection portion of the distal section of the delivery guide.

FIG. 11B is an enlarged view of section 812 in FIG. 11A.

FIG. 11C is an enlarged view of section 810 in FIG. 11A.

FIG. 11D is one embodiment of the power adapter illustrated in FIG. 3A.

FIG. 12 is a diagrammatic circuit illustrating power delivery for the electrolitically erodable section of the distal section of the delivery guide according to one embodiment of the invention.

FIGS. 13A-B illustrative side and perspective views, respectively, of another stent according to the invention.

FIG. 14A is a perspective view depicting another exemplary embodiment of a stent.

FIG. 14B is a perspective view depicting region 11B of FIG. 11A.

FIG. 14C is a schematic view depicting a cutting pattern for another exemplary embodiment of a stent.

FIG. 14D is a schematic view of region 11D of FIG. 11C.

FIG. 14 is a schematic view of a cutting pattern for another exemplary embodiment of a stent.

FIGS. 16A-B are illustrative views depicting exemplary power profiles for exemplary embodiments of the delivery system.

FIG. 17A is a perspective view depicting an exemplary embodiment of a precursor stent pattern.

FIG. 17B is a perspective view of region 13B of FIG. 13A.

FIG. 17C is a perspective view depicting another exemplary embodiment of a precursor stent pattern.

FIGS. 18A-B and 19 are schematic views depicting exemplary precursor stent patterns.

FIG. 20A is a schematic view of region 20A of FIG. 15.

FIG. 20B is a schematic view of region 20B of FIG. 16A.

FIG. 20C is a schematic view of region 20C of FIG. 15.

FIG. 20D is a schematic view of region 20D of FIG. 19.

### DETAILED DESCRIPTION OF THE INVENTION

### Angioplasty and Stenting Procedures

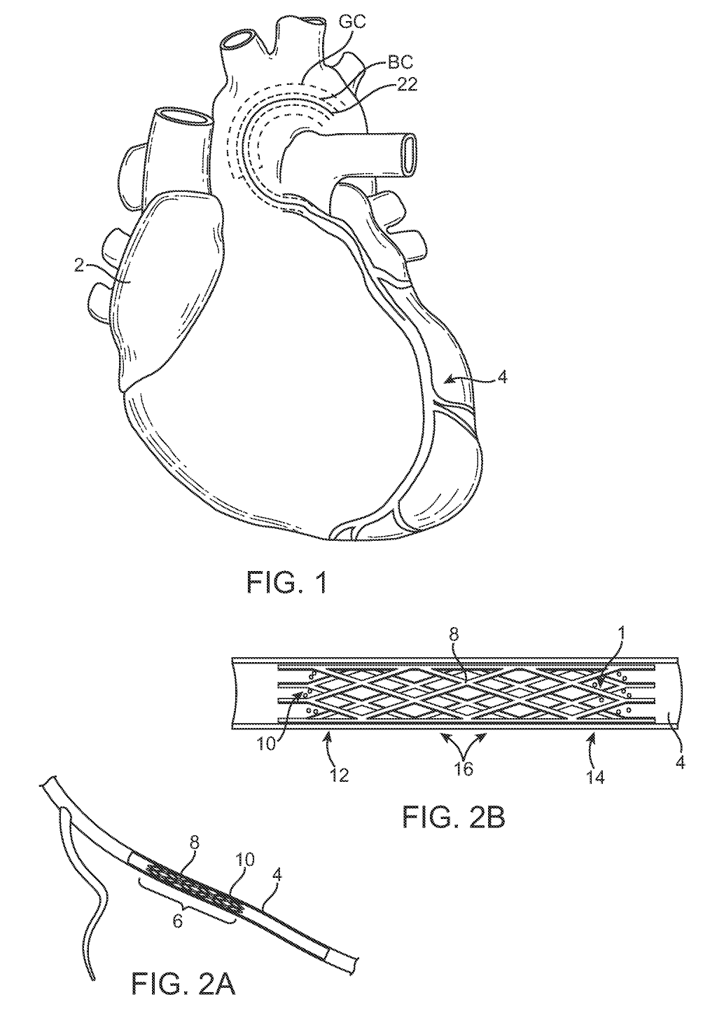

The devices, systems and methods described herein can be used for treating a heart 2 as shown in FIG. 1 by locating and releasing one or more stents within any of the coronary arteries 4. Stenting can be practiced in conjunction with angioplasty or “direct stenting” can be employed, where the stent can be delivered alone to maintain a body conduit, without balloon angioplasty. However, balloon predilatation and/or postdilatation at the site of the lesion to be treated can be employed. The balloon can be advanced to the site of the lesion prior to advancement of the delivery system or afterwards, in which case the delivery system can be used as a guide for the balloon catheter. Alternatively, the balloon can reside on the delivery system itself.

The system is advantageously sized for use in accordance with a “through-the-lumen,” methodology as described in U.S. patent application Ser. No. 10/746,455 “Balloon Catheter Lumen Based Stent Delivery Systems” filed on Dec. 24, 2003, which published as U.S. Patent Application Publication No. 2004/0193179 on Sep. 30, 2004, and its PCT counterpart PCT/US2004/008909 filed on Mar. 23, 2004, the disclosure of each of these references being hereby incorporated by reference herein in its entirety. The delivery guide can be capable of use as a lead guidewire suitable for over-the-wire or Rapid Exchange balloon catheter approaches. Alternatively, it can be substituted for a guidewire within the lumen of a balloon catheter as an intermediate step in an angioplasty procedure. Access to a treatment site is otherwise achieved with a collection of known devices in a manner routine to those with skill in the art.

In any case, after removal of the delivery guide from the treatment site as shown in FIG. 2A, the angioplasty and stenting procedure at the site of a lesion 6 within in vessel 4 may be complete. As detailed in FIG. 2B, an emplaced stent 8 and the desired resultant product in the form of a supported, open vessel in which plaque 10 may have been compressed through balloon dilatation remains. All of the near or proximal end 12, far or distal end 14, and a main body or support structure 16 of the stent extending there between is preferably in apposition with tissue or plaque at the site of the lesion.

In addition, other stenting endpoints may be desired such as implanting an anchoring stent in a hollow tubular body organ, caging or completely closing-off an aneurysm, delivering a plurality of stents and the like, when performing any of a variety of these or other procedures, suitable modification will be made to the subject methodology.

### Stent Design Overview

A “stent” as used herein includes any stent, such as coronary artery stents, other vascular prosthesis, or other radially expanding or expandable prosthesis, or scaffold-type implant suitable for the noted treatments and the like. Exemplary structures include wire mesh, ring or lattice structures. A “self-expanding” stent as used herein is a scaffold-type structure (serving any of a number of purposes) that expands from a reduced-diameter configuration (be it circular or otherwise) to an increased-diameter configuration. The mechanism for shape recovery can be elastic or pseudoelastic or driven by a crystalline structure change (as in a Shape Memory Alloy, i.e., SMA). While it is generally desirable to employ an alloy (such as nickel-titanium, or Nitinol alloy) set for use as a superelastic alloy, the material can alternatively employ thermal shape memory properties to drive expansion upon release.

Stents used with the devices, systems and methods described herein can be uniquely suited for a system able to reach small vessels (though use of the subject systems is not so-limited). By “small” vessels, it is meant vessels having an inside diameter from between about 1.5 to 2.75 mm and up to about 3 mm in diameter. These vessels include, but are not limited to, the Posterior Descending Artery (PDA), Obtuse Marginals (OMs) and small diagonals. Conditions such as diffuse stenosis and diabetes produce situations that represent other access and delivery challenges that can be addressed with the devices, systems and methods described herein. Other extended treatment areas addressable with the subject systems include vessel bifurcations, chronic total occlusions (CTOs), and prevention procedures (such as in stenting of vulnerable plaque).

A Drug Eluting Stent (DES) can be used in an application to aid in lessening late lumen loss and/or preventing restenosis. A review of suitable drug coatings and available vendors is presented in “DES Overview: Agents, release mechanism, and stent platform” a presentation by Campbell Rogers, MD incorporated by reference in its entirety. Examples of various therapeutic agents that can be used in or on the subject prosthesis include (but are not limited to) antibiotics, anticoagulants, antifungal agents, anti-inflammatory agents, antineoplastic agents, antithrombotic agents, endothelialization promoting agents, free radical scavengers, immunosuppressive agents, antiproliferative agents, thrombolytic agents, and any combination thereof. The therapeutic agent can be coated onto the implant, mixed with suitable carrier and then coated onto the implant, or (when the implant is made from a polymeric material) dispersed throughout the polymer. The agent can be directly applied to the stent surface(s), or introduced into pockets or an appropriate matrix set over at least an outer portion of the stent. The drug matrix, and/or even the stent itself, can be biodegradable. Several biodegradable matrix options are available though companies such as Biosensors International, Surmodics, Inc. and others. It is also recognized that bare-metal stents can also be employed.

In use, a self-expanding stent will typically be sized so that it is not fully expanded when fully deployed against the wall of a vessel in order to provide a measure of radial force thereto (i.e., the stent will be “oversized” relative to the vessel diameter). In a superelastic NiTi stent adapted for compression to an outer diameter of about 0.014 or about 0.018 inches and expansion to about 3.5 mm, the thickness of the NiTi can be between about 0.002 to about 0.003 inches (0.5-0.8 mm). Such a stent is designed for use in about a 3 mm vessel or other body conduit, thereby providing the desired radial force.

Such a stent can comprise NiTi that is superelastic at or below room temperature (i.e., as in having an Af as low as 0 to −15 degrees C.), or upwards of that, close to human body temperature (i.e., as in having and Af as high as 30 to 35 degrees C.). The stent can be electropolished to improve biocompatibility and corrosion and fatigue resistance. A binary alloy (i.e., NiTi—alone)—can be employed. Alternatively, various ternary alloys such as ones including chromium, platinum or other metals—for various reasons—can be employed.

Other materials and material procession approaches can be utilized for the stent as well. In addition to a drug or other coating or partial covering as referenced above, the stent can be coated with gold, palladium and/or platinum or any other biocompatible radiopaque substance to provide improved radiopacity for viewing under medical imaging. As practiced by Implant Sciences, Inc., a base layer of chromium can be desired to enhance adhesion of the more radiopaque metal layer(s). Various platinum or tantalum, etc. markers can additionally or alternatively be employed.

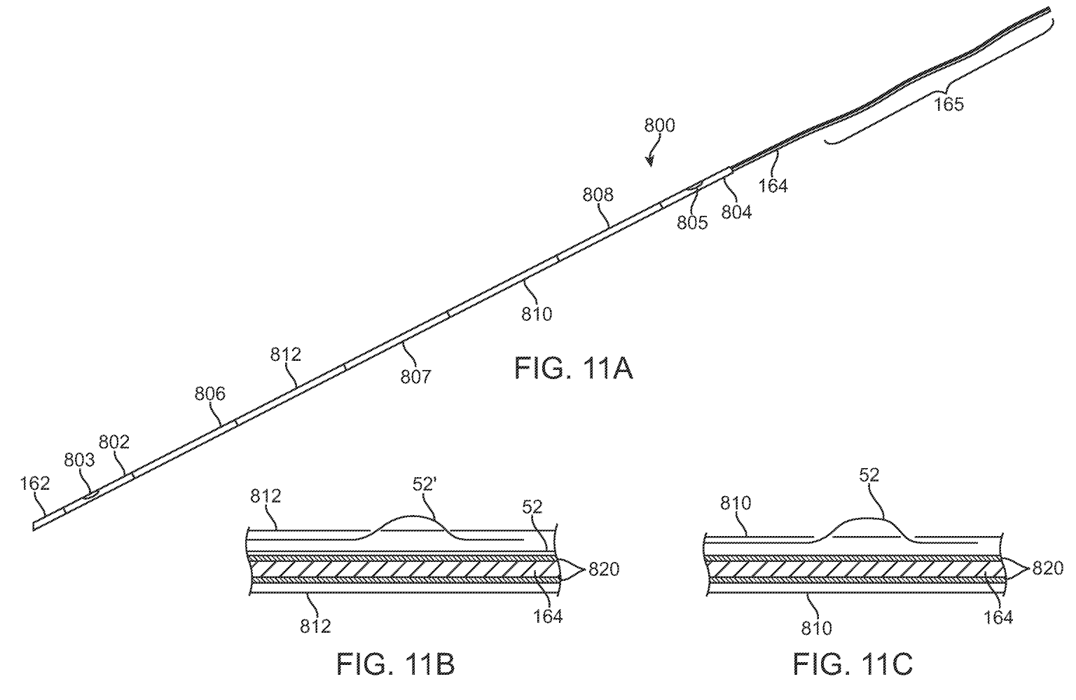

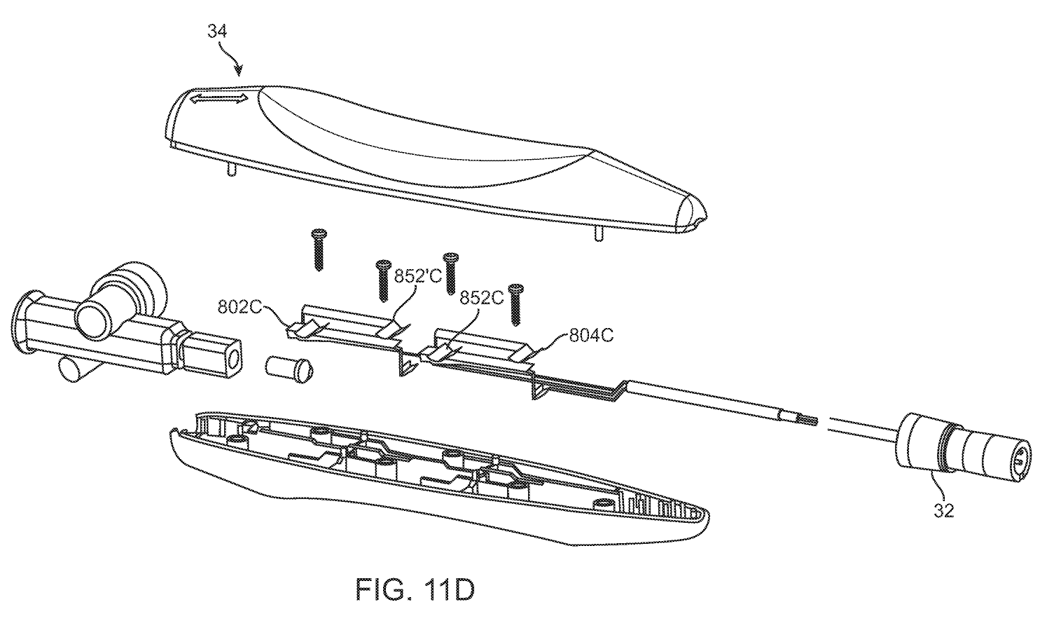

A superelastic nitinol (NiTi) stent for use with the devices, systems and methods described herein can be configured according to the cut pattern as taught in U.S. patent application Ser. No. 11/238,646 (pattern also shown in FIG. 11C), which published under U.S. Patent Application Publication No. 2006/0136037 on Jun. 22, 2006. Such a design is well suited for use in small vessels. It can be collapsed to an outer diameter of about 0.018 inch (0.46 mm), 0.014 inch (0.36 mm) or even smaller—and expanded to a size (fully unrestrained) between about 1.5 mm (0.059 inch) or 2 mm (0.079 inch) or 3 mm (0.12 inch) and about 3.5 mm (0.14 inch).

For use in twist-down type stent compression with delivery systems as described herein, end tabs or projections are typically provided. While straight projections are shown, others can be used as described in U.S. patent application Ser. No. 11/266,587, which published under U.S. Patent Application Publication No. 2006/0111771 on May 25, 2006, and U.S. patent application Ser. No. 11/265,999, which published under U.S. Patent Application Publication No. 2007/0100414 (the disclosure of each of these references being hereby incorporated herein by reference in its entirety) with complimentary seat/key features. The latter filing also describes in detail a manner of twist-loading stents as summarized below.

### Delivery System Overview

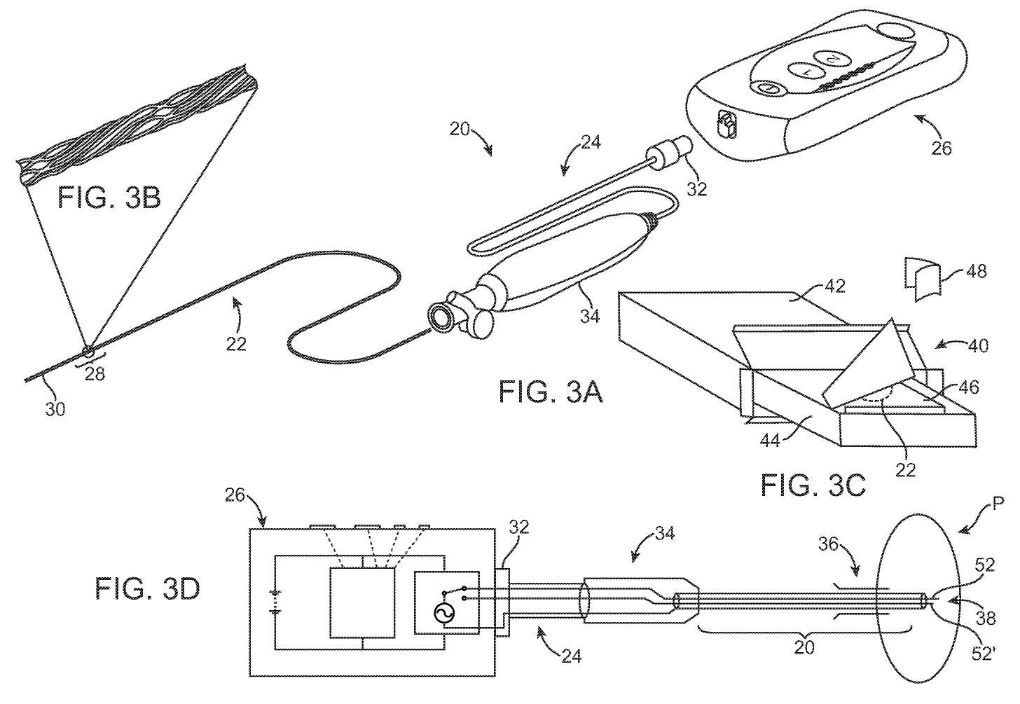

Referring to FIGS. 3A-C, an overview of an implant delivery system 20 for delivering stents optionally configured as described above is presented in FIGS. 3A-C. In this variation, delivery system 20 is shown including a delivery guide 22 and a power adapter 24, and a power supply 26. A distal section 28 of guide 22 carries a stent 8. The delivery guide 22 will typically terminate in an atraumatic coil tip 30.

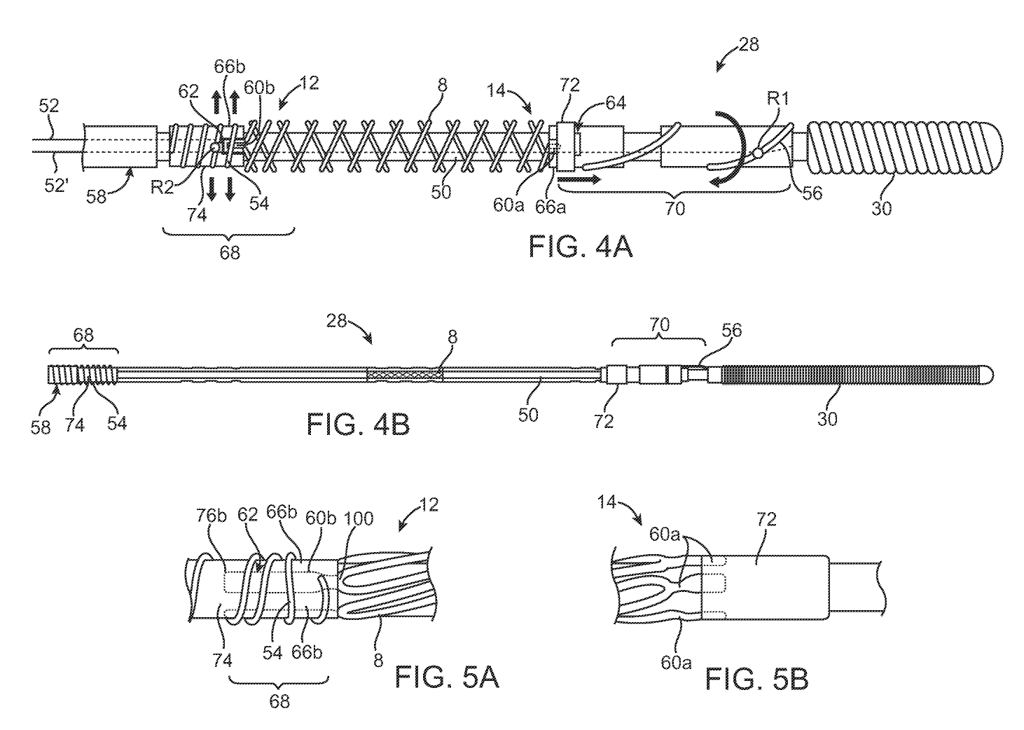

Referring to FIG. 3B, an enlarged stent section of distal section 28 is shown. As shown in FIG. 3C, the stent is held in a compressed diameter in-part by virtue of the twist imparted thereto. To release the stent, one or more latch members (shown in detail in FIGS. 4A-5B) are eroded by application of voltage via power supply 26.

Electrolytic erosion of a bare/exposed metal latch section is driven by applying voltage to develop a positive charge on the element resulting in a motive force to cause current to flow to a (relatively) negatively charged body (e.g. a neutral pole). Current flows by ion transfer from the section to be eroded to the negative body through an electrolytic solution. Within a patient, the solution is the patient's blood. Further discussion of electrolytic detachment/release is presented in various patents including U.S. Pat. No. 5,122,136 to Guglielmi; U.S. Pat. No. 6,716,238 to Elliot; U.S. Pat. No. 6,168,592 to Kupiecki, et al.; U.S. Pat. No. 5,873,907 to Frantzen and the multiplicity of continuation, continuations-in-part and divisional applications related to these patents.

Power supply 26 incorporates a circuit board and one or more batteries (e.g., lithium ion “coin” cells or a 9V battery) to provide power to the system's features to selectively drive the erosion. The power supply shown is reusable. It will typically be bagged (bag not shown) within an operating room. A disposable power adapter/extension 24 including appropriate connectors 32 and a handle interface 34 is provided in sterile packaging 40 with the delivery guide 22.

FIG. 3D provides a schematic illustration of the electrical hardware shown in FIG. 3A. An introducer catheter 36, the patients body “P” and electrodes 38 in contact therewith are additionally illustrated.

The packaging can include one or more of an outer box 42 and one or more inner trays 44, 46 with peel-away coverings as is customary in medical device product packaging. Instructions for use 48 can also be provided. Such instructions can be printed on the product included within packaging 40, printed on a sheet of paper, or be provided in another readable medium (including, but not limited to a computer-readable medium). The instructions can include provision for basic operation of the subject devices and associated methodology.

In support of implant delivery, it is also to be understood that various radiopaque markers or features can be employed in the delivery system to (1) locate implant position and length, (2) indicate device actuation and implant delivery and/or (3) locate the distal end of the delivery guide. As such, platinum (or other radiopaque material) bands, use of such material in constructing various elements of the subject systems, and/or markers (such as tantalum plugs) can be incorporated into delivery 22 guide itself.

In one exemplary embodiment, delivery systems are advantageously sized to match the diameter of a commercially available guidewire. In the most compact variations, the delivery guide has an effective diameter that can range from 0.014 inch (0.36 mm) up to and including 0.018 inch (0.46 mm). However, the system can even be advantageously practiced at 0.022 inch (0.56 mm) or 0.025 inch (0.64 mm) sizes. Of course, intermediate sized wires can be employed as well, especially for full-custom systems.

In smaller sizes, the system is applicable in “small vessel” cases or applications or treatment. In larger sizes, the system is most applicable to larger, peripheral vessel applications, biliary ducts or other hollow body organs. The latter applications involve a stent emplaced in a region having a diameter from about 3.5 to 13 mm (0.5 inch). In any case, sufficient stent expansion is easily achieved with prostheses employing the features of the exemplary stent pattern shown.

### Delivery Guide Feature Details

While FIG. 3A illustrates a full-size delivery system, a number of the following figures illustrate detail views of the far or distal end 28 of such a system. This “working” or active end is incorporated into complete systems and can be used in the manner described, as well as others as may be apparent to those with skill in the art. The constituent parts of the systems include structural wire, hypotubing sections and electrical leads as further described or processed (such as by taper grinding, etc.) as those with skill in the art will appreciate.

Structural “wire” used herein generally includes a common metallic member such as made of stainless steel, NiTi or another material. The wire can be at least partially coated or covered by a polymeric material (e.g., with an insulating polymer such as Polyamide, or a lubricious material such as TEFLON®, i.e., PolyTetraFluoroEthelyne or PTFE). Still further, the “wire” can be a hybrid structure with metal and a polymeric material (e.g., Vectran™, Spectra™, Nylon, etc.) or composite material (e.g., carbon fiber in a polymer matrix). The wire can be in the form of a filament, bundle of filaments, cable, ribbon or in some other form. It is generally not hollow. The wire can comprise different segments of material along an overall length. “Hypotube” or “hypotubing” as referred to herein means small diameter tubing in the size range discussed below, generally with a thin wall. The hypotube can specifically be hypodermic needle tubing. Alternatively, it can be wound or braided cable tubing, such as provided by Asahi Intec Co., Ltd. or otherwise. As with the “wire” discussed above, the material defining the hypotube can be metallic, polymeric or a hybrid of metallic and polymeric or composite material. Solder, welding (e.g. resistance or laser) or glue (e.g., standard medical-use epoxy or UV cure) can be used to secure the various material sections shown.

As shown in FIG. 4A, working end 28 carries stent 8 held in a compressed diameter over a hypotube section 50. Electrical leads 52 and 52′ pass through the hypotubing. Proximal latch wrap 54 is electrically connected to lead 52, while 52′ is electrically connected to distal latch wire 56. Lead 52 and wrap wire can comprise a single length of wire, or pieces such as copper for the lead and stainless steel for the wrap that are connected/soldered together; the same holds true for lead 52′ and latch wire 56.

However configured, leads 52/52′ can be employed for connection to discrete channels or circuits (in combination with a return lead/path as can be provided by hypotube 50 and/or hypotube delivery guide body 58, a specialized catheter, for example, as described in U.S. Pat. No. 6,059,779 to Mills, or an external pad placed upon a patient's body, for example as described in U.S. Pat. No. 6,620,152 to Guglielmi) to provide individual control over corrosion of the wires. Such a setup can be desired in order to first release the distal side of the implant and then release the proximal side.

Further, latch action can be monitored. When current no longer flows on a given circuit, positive indication is offered that the subject latch has been released. Another beneficial factor is that by eroding one latch at a time, current can be limited, in contrast to a system in which multiple sections of material would be eroded at once. The current draw necessary to erode the subject latches is also minimized by controlling latch size.

The latch wires shown are insulated except for a sacrificial material section or region “R”. To define the sacrificial region, polyimide insulation or a protective layer of noble (or more noble) metal such as platinum or gold covered other portions of the material is stripped off, removed, or never laid-down in the first place via a masking process at the section. Stainless steel wire will generally be selected for its strength and because it offers corrosion resistance “on the shelf” while being erodable in an electrolytic solution under power. Other material selection and construction options as discussed in the incorporated references are possible as well.

Precisely manufactured latch regions can be produced using a laser to ablate insulation on wire over a selected region. Such an approach is advantageously employed to provide erodable exposed wire section(s) having a length as little as about 0.001 inches long. More typically, latch length ranges from about 0.001 to about 0.010, preferably between about 0.002 and about 0.004 inches on a wire having a diameter between about 0.00075 and about 0.002 inches. Insulation thickness can be as little as about 0.0004 to about 0.001 inches, especially when an intermediate protective polymer layer is employed in the latch assembly as described in further detail herein. Its thickness can fall outside this range as well—as can other dimensions not indicated as critical herein.

Delivery guide portion 28 shown in each of FIGS. 4A and 4B employs a stent 8 including projections 60 that are adapted for sliding receipt with near and far seat features 62 and 64, respectively, defined (at least in part) by key “fingers” 66. In FIG. 4A, a twist imparted to the stent to hold the same in a compressed state (i.e., without a medial covering) is indicated by different weight hatching. In FIG. 4B, it is indicated by hatching across a straight-compressed stent. The twist imparted to an actual stent and overall proximal and distal latch assemblies 68 and 70, respectively, are shown in close-up schematic images in FIGS. 5A and 5B. The latch architecture regions are also indicated in FIGS. 4A and 4B.

However pictured, to release stent 8 with a combination of latches as shown, distal latch wire 56 is first eroded. Breaking this member allows seat 64 to rotate together with connected sleeve section(s). Optional blocker underlying the sleeve sections restrains axial movement of the latch elements. Further discussion of the latch architecture is presented in above-referenced U.S. patent application Ser. No. 11/265,999.

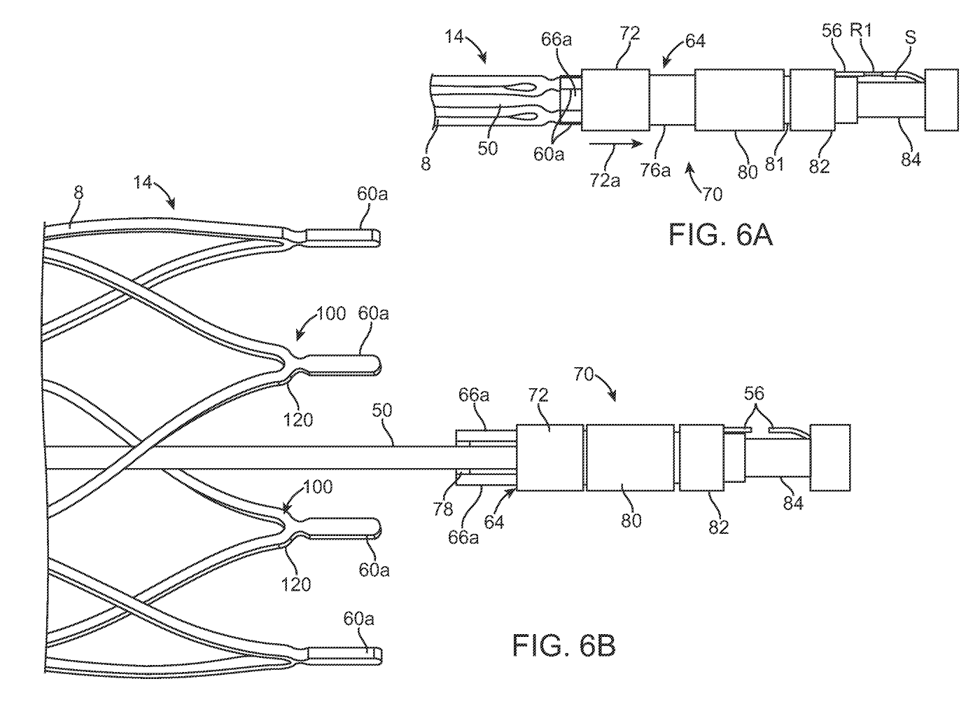

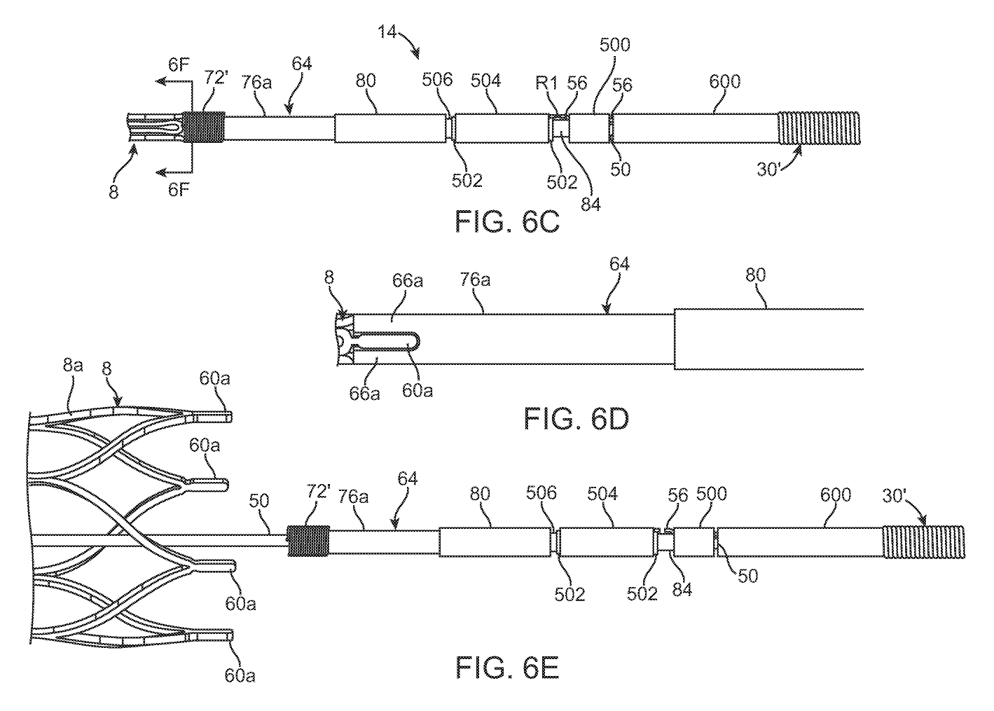

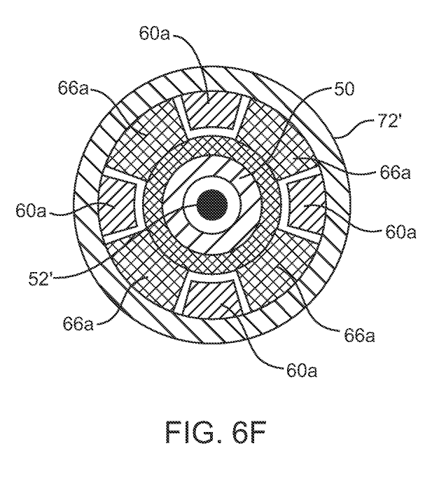

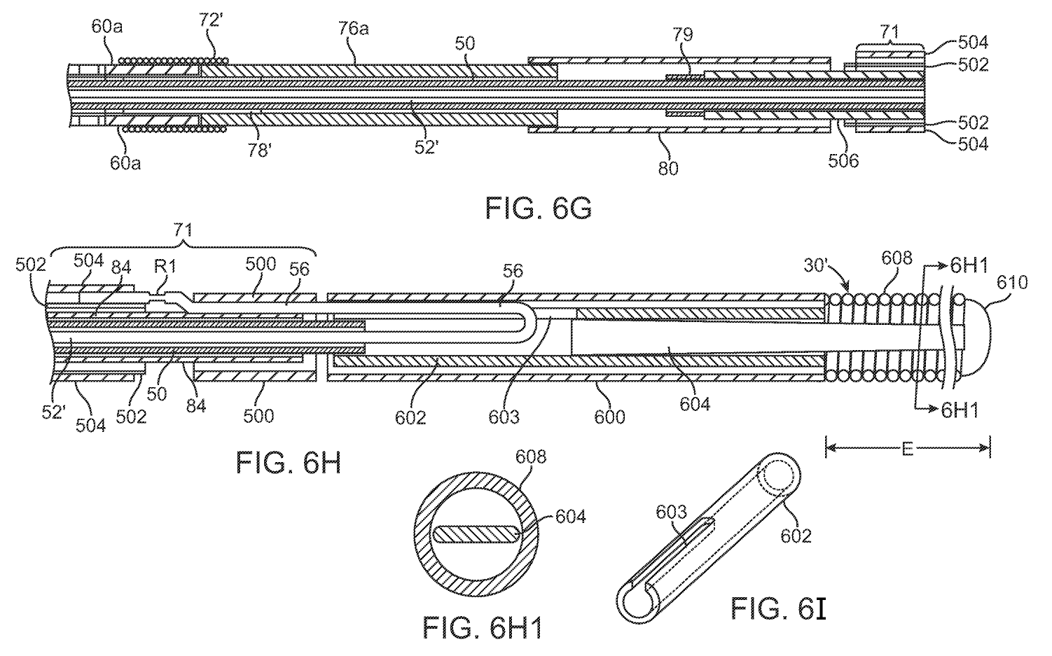

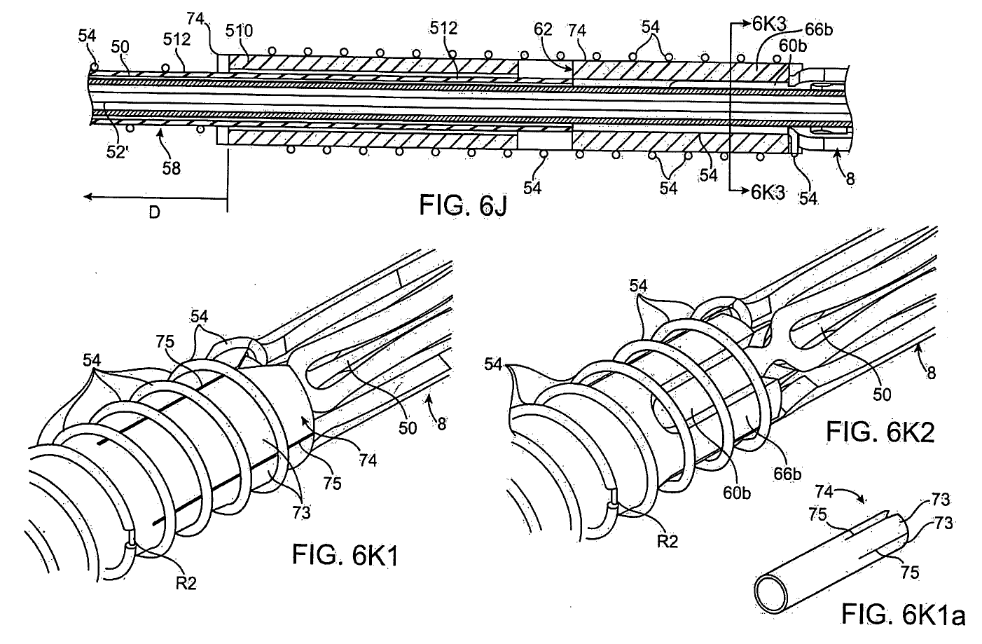

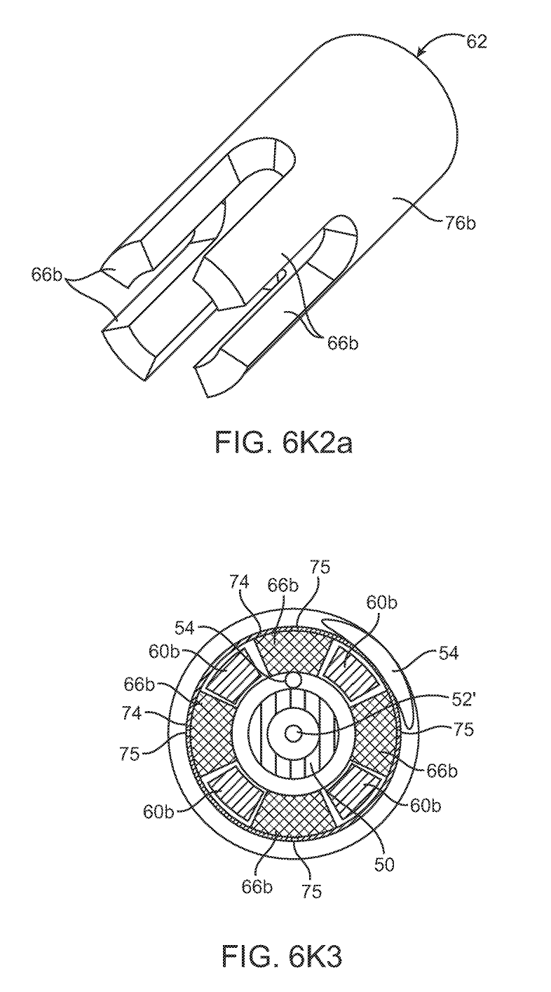

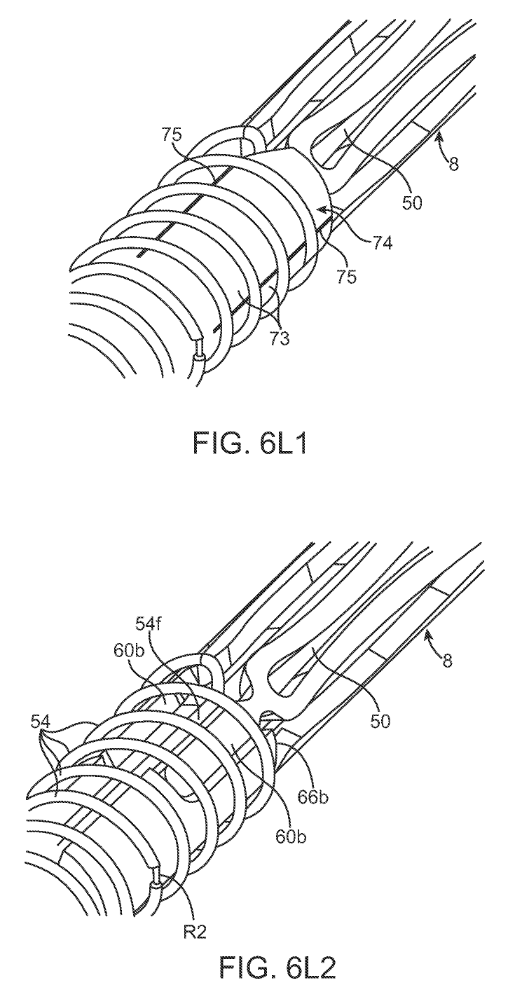

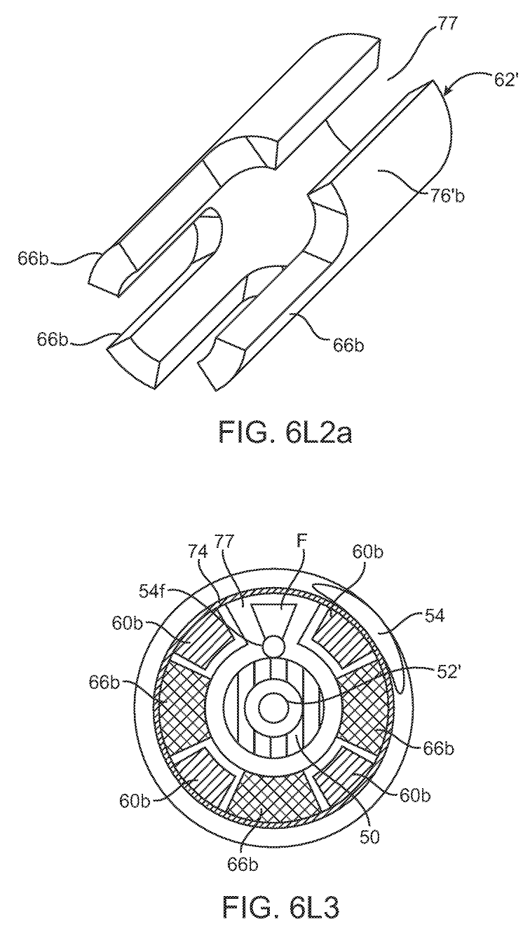

However it is specifically configured, release of the rotatable assembly allows the associated stent to untwist and expand. The expansion results in foreshortening that pulls the stent's distal projections from seat 64. Also, radial expansion of the stent forces “floating” (i.e., unsecured) band 72 to be driven off the stent projections 60 along fingers 66, away from a capture/capturing configuration in conjunction with overall seat 64. Such action is depicted in FIGS. 6A and 6B.

Returning, however, to the variation shown in FIG. 6B, without the stent obscuring the view, the figure also shows an optional stabilizer band 78 to which the key fingers can be welded or originally attached through construction as a one-piece assembly. Band 78 can be of use in stabilizing the position of the key fingers when loaded by virtue of interaction with the stent tabs/projections. In addition, use of such a band allows the tabs to achieve a greater angle of action upon slider 72 for driving it distally upon initial stent expansion during untwist to promote release.

However, when slider band 72 is fixed in position, band 78 can more advantageously be omitted. In this case, foreshortening of the stent upon untwisting/unwinding causes release from seat 64.

Not shown (in either case) is a blocker type feature that underlies connection sleeve 80 securing seat body 76 to hub 82 through tube 81, where the blocker maintains the axial position of the latch assembly (i.e., except slider band 72) upon release. Soldering or welding can be employed for attachment between metal members, and/or glue (typically epoxy) joints used throughout.

And while the blocker is adapted to limit proximal axial movement of the overall latch assembly 70, it can be configured to allow some lateral play in the assembly (e.g., about 0.020 inches for constructing an 0.014 to 0.018 inch diameter system) for assembly purposes. The blocker can be integrally formed by step-grinding a feature in hypotube 50 or comprise a band (metal or polymeric) affixed thereto. An additional polymeric insulator layer 84 can be provided underneath the latch to improve electrical robustness such that the system does not short out despite gap or space “S”.

Sleeve 80 preferably (though not necessarily) includes stainless steel in order to offer both greater structural integrity given that it transmits load from the stent to latch wire 54 until release. A metal sleeve 80 also advantageously serves to bring the return path of the circuit closer to the erodable region R, given that sleeve 80 is in electrical contact with metal seat 64 (possibly also metal blocker) which is/are in electrical contact with metal hypotube that completes the electrical circuit.

Stabilizer band 78 can comprise stainless steel; preferably it is seamless tubing. In one variation, it includes electroformed Nickel-Cobalt alloy. Such material is initially deposited on an aluminum mandrel that is subsequently etched away once the part has been cut to length. Wall thickness of either material can range from about 0.00075 to about 0.0015 inches or more. Another processing variation contemplated (whether a steel or Ni—Co band is employed) is reinforcement of the tubing by a coating layer of epoxy. Yet another processing variation involves use of a laser to alter material properties around the edge at which the stent tabs and slider band interface (i.e., the proximal or leading edge in the delivery system variation shown). Yet another option contemplates producing a slider band as described in U.S. patent application Ser. No. 11/147,999 or its corresponding WO counterpart filed Jun. 7, 2006.

As with the “untwisting” type latch assembly, the basic architecture of the proximal wrap-type latch assembly 68 shown in FIGS. 4A, 4B and 5A is described in U.S. application Ser. No. 11/265,999. Specifically, wire or ribbon 54 is wrapped over or around proximal stent tabs 60 and seat 62. As such, a positive lock on the implant until proximal release is desired. Therefore, the system can be withdrawn (with the stent attached thereto) most easily in case emergency withdrawal is required. In other words, by including a wrap over the interlocking or interfitting features, their orientation is stabilized relative to opposing surfaces.

Wrap member 54 also includes an erodable section “R”. Upon release of the wire or ribbon wrap by erosion of the erodable section, the wrap wire/ribbon at least partially unwinds or unravels to allow projections 60 to translate out of the their complementary seat features. In other words, stent release with a wrap-type latch assembly as shown in each of FIGS. 4A, 4B and 5A rely upon the wrap loosening to release a hold on stent tabs 60 captured within complementary seat regions 62. Such action is depicted in FIG. 4A by the outwardly-directed arrow.

In wrap-type retention and release assembly 68, between two and four wraps of the wire or ribbon 54 over the stent tabs 60 are advantageously employed in a system capable of being sized to an outside diameter or crossing profile of about 0.014 inches. The location of the erodable section of the latch is advantageously set outside of the stent extension/tab region. This way, the possibility of twisted stent tabs coming into contact with the exposed latch region “R” (shorting-out the system) is minimized—thereby contributing to electrical robustness.

Yet, the erodable section should not be removed from the region to be released by too many wraps. With successively more than four wraps (i.e., in an 0.014 inch-compatible delivery system) potential for binding or incomplete release is increased such that release is not ensured.

Even with such a wrap approach, the latch configuration advantageously employs an insulation layer intermediate to the wrap wire or ribbon 54 and seat member 62. Such an insulation layer 74 can vary in construction as summarized above. As shown in detail in FIG. 5A, however, polyimide tubing is provided over seat body 76 and fingers 66. It is scored or slit to facilitate the stent projection escape from the seat region.

So-cut, the polymeric insulation layer offers little or no significant impediment to stent release. However, it offers an appreciable improvement in system electrical robustness. Because latch wire 54 is tightly wound about the delivery guide seat, without the polymer layer it is difficult to ensure (i.e., at least without significant qualification testing) that the section exposed for erosion “R” will not short-out by contact with the seat region, that twisted tabs cut through insulation of the latch wire, or the erosion region R contact the stent tabs when it is located along their length.

Most advantageously, cut open or operable sections interposed insulation layer 74 are axially aligned with or run along the seat fingers 66. In this manner, any twist or non-uniformity of loaded tab features will not (or cannot) force their way into contact with the latch wire 54 though its thin insulation or with the exposed erodable section R should it be located in the region of the tabs. Still (especially in connection with the offset tab features described below), insulation layer 74 can offer advantages however it is oriented radially in terms of overall system electrical robustness.

Indeed, such advantages can be achieved with a polymer “short cover” 74 that adds very little to the overall diameter of the system. One material that can be employed is polyimide tubing. The material thickness can be as little as 0.005, 0.001, 0.0005 inch wall thickness or less. Naturally, other polymers or wall thicknesses can be employed. However, for the smallest delivery system diameters, material thickness is minimized. Whatever the material selected, the sleeve or cover is advantageously bonded or otherwise to the underlying seat to ensure its proper location during and after system assembly.

Referring to FIG. 6C-F, another embodiment is shown where restraint 72 is replaced with restraint 72′, which is in the form of a wire coil. Coil 72′ covers axially extending distal tabs or projections 60a, which extend from stent body 8a of stent 8, as shown in FIGS. 6C and D. Stent 8 has a closed cell construction as shown, for example, in FIGS. 2B, 6B, and 6E, 3A and 13B. Such a closed cell construction is a non-coil type construction where the stent struts or wire form closed cells. As in the case with tubular restraint 72, restraint 72′ keeps tabs 60a seated in seat 64 (see e.g., FIG. 6F, which is a transverse sectional view taken through restraint 72′) and keeps the tabs from radially expanding, and thus the stent in a compressed state for low profile delivery.

In this embodiment, the overall release mechanism comprises distal latch assembly 71 and a key assembly. Distal latch assembly 71 includes tubes 504. 502, 500, and 84 and wire 56 with erodable or sacrificial portion R1. The distal key assembly includes members 64, 72′, 78′, and 79, and latch mount 506.

Referring to FIGS. 6G and 6H, the distal key and latch assembly will be described in further detail. Distal coil band 72′, which can be made out of 0.0012 inch wire, is laser welded to distal fingers 66a of seat 64, which is soldered to tubular connector tube 80. Tubular connector is soldered to tubular latch mount 506. In this manner, sleeve 80 connects seat body 76a to hub or tubular member 506, which extends into tubular member 502, which is surrounded by tubular member 504. Tubular stabilizer 78′, which can be, for example NiCo, is slidably positioned around central tube 50 and slidably positioned within the inner perimeter of tabs 60a and seat fingers 66a such that it can freely float or slide. Stabilizer band 78′ provides support for tabs 60a and friction reduction to facilitate stent deployment. Tubular blocker 79 is soldered to central tube or twist mandrel 50 and is sized to prevent latch mount 506 and all elements fixedly secured to latch mount 506 (i.e., members 64, 72′, and 80 and distal latch assembly 71) from moving proximally. Tubes 502 and 504 are not secured to the twist mandrel 50 so they can rotate as the stent untwists

Distal latch assembly 71 is epoxied to latch mount 506 by epoxying tube 502 over latch mount 506. Tube 502 is bonded to tube 504 and at the same time wire 56 is bonded between tubes 502 and 504. Tube 502 is bonded over latch mount 506 and the distal end of 84 is bonded to the twist mandrel 50.

Coil band 72′, distal key 64, tube 80, tubular latch mount 506, tubular blocker 79, and central tube or twist mandrel 50 can comprise stainless steel to provide and electrically conductive pathway for ground. An additional layer of insulative material such as insulative tube or sleeve 84 can be provided between wire 56 and central tube 50 to provide additional protection against shorting between wire 56 and central tube 50.

Referring to FIG. 6H, atraumatic coil tip 30′ can comprise a tip coil 608 having a rounded distal end or solder ball 610 and a core wire 604 extending through the tip coil and attached or extending from rounded distal end 610. Tube 602 secures tip coil 608 to central tube or twist mandrel 50. Tube 602 includes a slot 603 that opens at the proximal end of the tube to provide a passage for wire 56 so that the wire can pass through tube 602 after exiting tube 50 and then extend proximally where it passes between tube 500 and sleeve 84 and then between tube 502 and sleeve or cover 504. The distal end of wire 56 is secured to twist mandrel 50 as well as to insulative tube 600 and slot 603 in tube 602 by applying and curing epoxy. Tube 602 is bonded (e.g., with solder and epoxy) to central tube or twist mandrel 50 and soldered to tip coil core wire 604. Tip coil 608 is soldered to tip coil wire 604 and to the distal end of tube 602. An insulative tube or sleeve 600 is then positioned over tube 602 and a distal portion of distal latch wire 56 to surround tube 602 and wire 56. The sleeve is secured to tube 602 and wire 56 with, for example, epoxy. In one embodiment, tip coil 608 is platinum, core wire 604 is stainless steel, insulative tube 600 is polyimide tubing, and distal latch wire 56 is polyimide coated stainless steel wire.

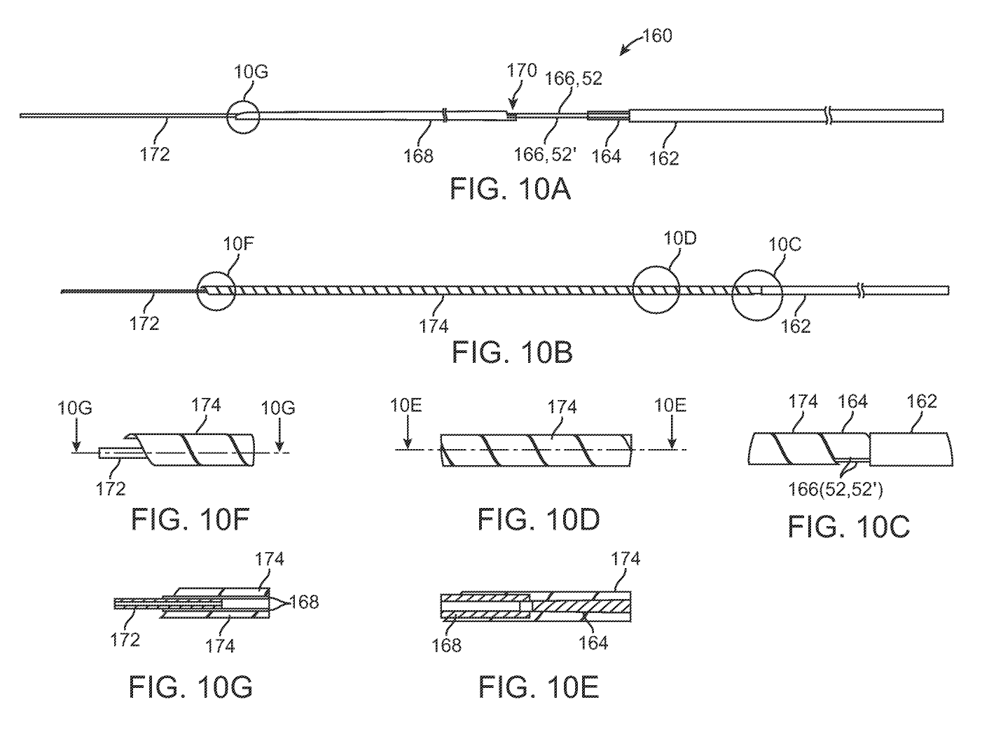

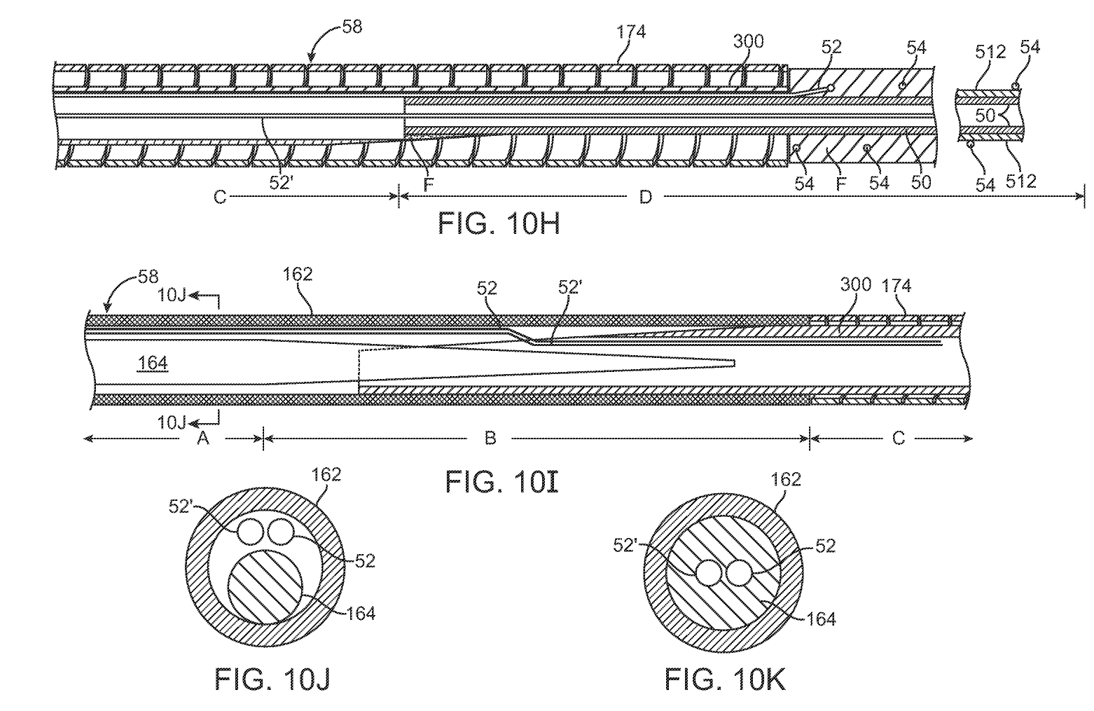

Referring to FIG. 6J, a sectional view of another embodiment of the proximal latch assembly, which releasably holds the proximal end or tabs 60b of stent 8 in a radially compressed state, is shown. In this embodiment, the proximal latch assembly includes a stent seat 62 having a plurality of fingers or projections 66b between which are seated stent tabs 60b. An insulative sleeve or tubular member 512 surrounds central tube 50 and extends about 1-2 mm proximal to marker 510 and is spaced from member 174. Insulative sleeve 512 can comprise polyimide tubing. A radiopaque marker can be provided proximal and adjacent or close to stent seat 62 to provide an indication of the location of the proximal end of the stent during delivery. In the embodiment illustrated in FIG. 6J, a radiopaque marker is shown and designated with reference numeral 510. Marker 510 can be a tubular member, which surrounds central tube 50. It can be made of any suitable material such as platinum. The portion of proximal latch wire 54 (which can be polyimide coated stainless steel wire) that is proximal to erodable portion R2 (which like erodable portion R1 can have a length of 0.002-0.005 inch) is bonded (e.g., with epoxy) to insulative sleeve 74 then wraps around the section with stent tabs 60b, and then goes under proximal key or seat 62, which can be stainless steel. Filler F, which can be epoxy, extends from helically wound ribbon tubing 174 (FIG. 10H) to tubular member 74 (FIG. 6J).

Wire 54 wraps around insulative sleeve 74 and insulative polyimide tubing 512. The length of proximal latch wire 54 over proximal platinum marker 510 and insulative polyimide tubing 512 is bonded with epoxy to insulative sleeve 74 and insulative polyimide tubing 512. The portion of 54 distal to sacrificial link R2 is not secured to insulative sleeve 74. Typically, about 1-5 helical turns of helical wrap wire 54 immediately proximal to noninsulated sacrificial link R2 are not secured to insulative sleeve 74, insulative tubing 512, or marker 510. However, the remaining portion of the wrap is secured to the material which is surrounds (insulative sleeve 74, insulative tubing 512, or marker 510). Stainless steel proximal key 62 is bonded (epoxy) to stainless steel twist mandrel 50.

In the embodiment illustrated in FIGS. 6K1,6K2, and 6K2a, four stent tabs 60b are used, each being seated between an adjacent pair of the four seat fingers 66b of the four finger seat body 62 embodiment best shown in FIG. 6K2a.

Referring to FIGS. 6K1 and 6K2, FIG. 6K1 shows optional insulative sleeve 74 positioned between proximal latch wrap wire 54 (which is the helically wound portion of lead 52) and stent tabs 60b, while FIG. 6K2 shows insulative sleeve 74 removed to illustrate one of the tabs 60b (the others are hidden from view). In this embodiment, insulation layer or sleeve 74, which can be in the form of a polyimide tube, includes a plurality of slits 75, which extend to the distal end of tubular sleeve 74. The slits are positioned over or close to seat fingers 66b so that stent tabs can pass therethrough as portions 73 between slits 75 can move radially after the wire wrap electrolytic sacrificial portion R2 erodes allowing the proximal portion of the stent with it tabs to radially expand. Sacrificial or erodable portion R2 typically is positioned as close as possible to the tab 60b closes thereto or within about three wrap turns of the tab to minimize the amount of wrap that may remain coupled to fingers 66b.

Although a four tab and finger configuration is illustrated, other numbers of tabs and fingers can be used. In one variation, latch wire 54 can form one of the fingers as shown in FIG. 6L1 and 6L2. With this configuration, a seat body such as seat body 62′ as shown in FIG. 6L2a with three fingers is used.

A transverse cross-section of the four finger embodiment of FIG. 6K1, 6K2, 6K2a is shown in FIG. 6K3 and a transverse cross-section of the three finger embodiment of 6L1, 6L2, and 6L2a is shown in FIG. 6L3. In the three finger embodiment shown in FIG. 6L3, wire 54 is positioned in the place of the missing finger and that portion of the wire is referred to wire finger 54f. Filler “F” can be provided over wire finger 54f to provide an enhanced seat for the corresponding tabs 60b that are to be placed between the fingers that are circumferentially nearest to wire finger 54f. Slot 77 is formed in seat body 76′b to allow wire finger 54f to pass therethrough and be secured to tube 50 and seat 62′ with any suitable means such as epoxy.

### Stent Loading

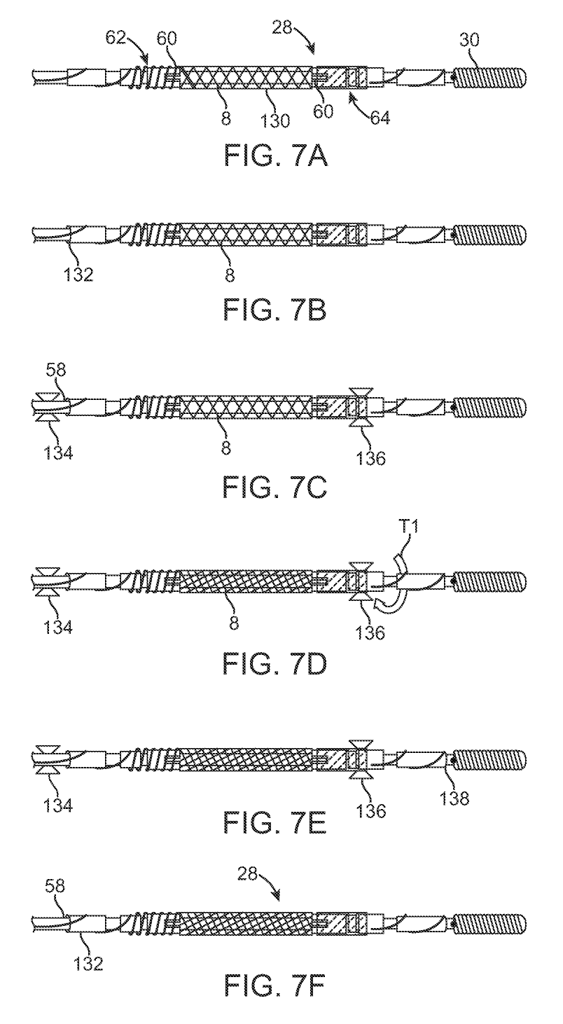

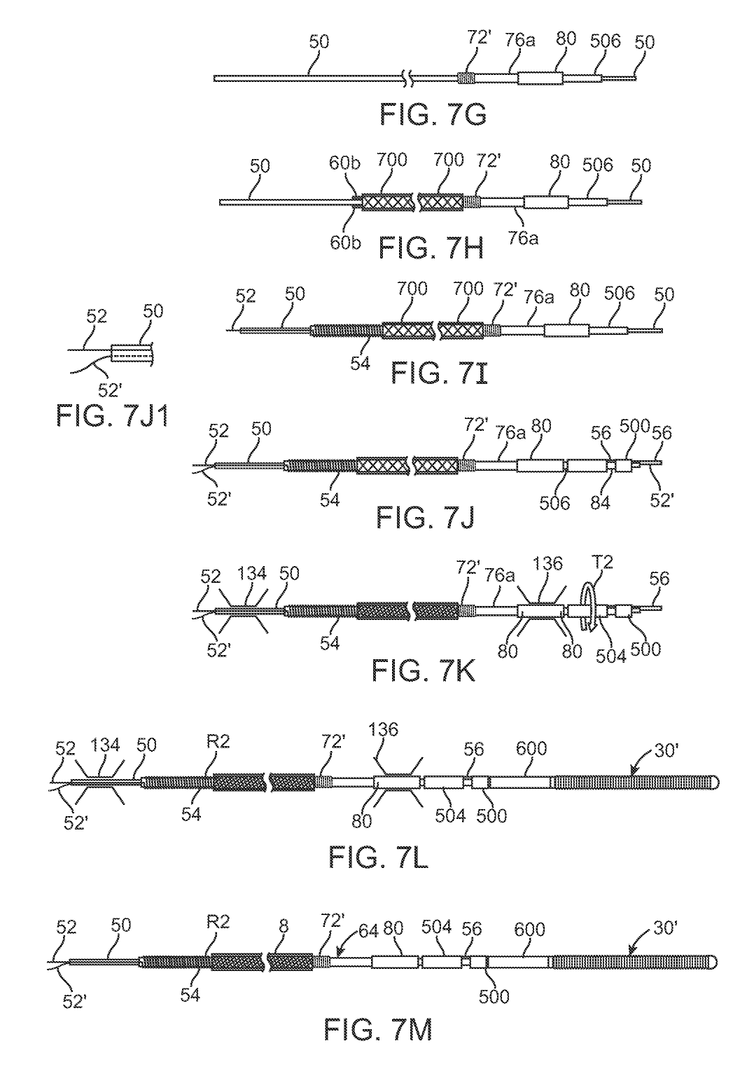

FIGS. 7A-7F show an approach to loading a stent onto a delivery system. In this method, the stent is compressed by hand, with an automated “crimper” such as produced by Machine Solutions, Inc., or otherwise, without a substantial twist imparted thereto. The stent can be compressed by virtue of the act of loading it into a tube, or loaded into a tube after being compressed by a machine. In any case, the tube or sleeve that it is loaded into will generally be close in diameter to its final size when secured upon or the delivery guide. By “close” in diameter, what is meant is that it is within at least about 33%, or more preferably within about 25% to about 10%, or even within about 5% or substantially at its final diameter. Then, with the stent so constrained, it is twisted from either one or both ends before of after partial or full attachment to the delivery guide.

The sleeve can comprise a plurality of separate pieces or segments (most conveniently two or three). As such, the individual segments can be rotated relative to one another to assist in twisting the stent. In addition, axial manipulation of the relation of thin individual segments can be employed to allow the implant to bulge outwardly over one section. The foreshortening caused by this action can then allow positioning and then axially loading end interface members by manipulating the segments to collapse the bulging.

The figures illustrate a process of loading a delivery guide using only a single restraint sleeve. To carry out the additional acts above, or to reduce the degree to which the stent must twist inside a single sleeve, sleeve 130 can be broken into a number of segments (before or after loading a compressed stent therein) as indicated by broken line.

As for the specific example of loading, FIG. 7A shows stent 8 captured within a temporary restraint 130 and set over a delivery guide distal section 28. Its placement therein causes the stent to lengthen to about its full extent. The stent 8 includes projections 60 serving as near and far mating portions interfacing with proximal seat and distal seat features 62 and 64, respectively. Each of the seats can—at first—be free to rotate.

FIG. 7B shows a first glue or solder joint 132 laid-down to secure one of the seats from rotating. While the near seat 62 is the one secured, either one of them can be. The approach shown here is merely intended to be illustrative. Indeed, this process step or others can be altered without departing from the general approach.

Returning to the illustrated method, however, FIG. 7C illustrates clamp members 134 and 136 grasping portions of the delivery guide. The near clamp 134 grasps the body 58 of the delivery guide and the far clamp 136 holds structure associated with the far seat 64.

The clamps can comprise part of a simple twist fixture supporting chucks aligned on bearings, etc. In any case, in FIG. 7D, the clamps are rotated relative to one another (in the example illustrated, only the distal clamp is rotated because the proximal one is held stationary). As indicated by the change in the illustrated structure, a twisted stent form 8′ now lays underneath restraint tube 130.

Following the twisting of the stent within the tube, the distal seat 64 is secured from counter rotation by a glue or solder joint 138. Finally, clamps or chucks 134 and 136 are released and restraint 130 is cut, peeled or slid off of the delivery guide body 58 to ready the system for stent deployment as shown in FIG. 7F.

Note, however, that the act of restraint 130 removal can take place even in the operating room as a final step prior to delivery guide use. Otherwise, it can occur as some step along the manufacturing process. When employed in the former manner, sleeve 130 will then do double duty as a loading and a storage sleeve.

Referring to FIGS. 7G-M, another method of stent loading or assembling the delivery guide will be described.

Referring to FIG. 7G, center tube 50 is provided with distal seat body 76a from which fingers 66a extend and to which connector tube 80 is fixedly secured. Connector tube 80 is fixedly secured to tubular latch mount 506.

Referring to FIG. 7H, stent 8 is then radially compressed and introduced into one or more sleeve(s) 700 depending on the length of the stent. Distal tabs 60a are seated between fingers 66a and under coil 72′ slid over the tabs to prevent them from radially expanding.

Referring to FIG. 7I (and FIGS. 6K2A and 6K3), proximal seat 62, which comprises proximal seat body 76b and fingers 66b, which extend from proximal seat body 76b, is slid over central tube 50 this is done after the above-referenced distal latch components are mounted on central tube 50. Seat 62 is slid on 50 from the proximal end of 50. Insulative sleeve 74 is slid over seat 62 and wire 52 wrapped around seat 62 to form wire wrap 54 and the distal end of wire 52 secured to seat 62 to restrain tabs 60b and prevent them from expanding radially outward. FIG. 7J1 is an enlarged view of the proximal end portion of the apparatus shown in FIG. 7J.

Referring to FIG. 7J, the tubes of latch assembly 71 (tubes 500, 502, 504, and 84) are added and wire 56 is extended proximally through tube 50 where it is referred to as lead 52′.

Referring to FIG. 7K, proximal clamp 134 is used to clamp a portion of the delivery guide proximal to proximal seat 62 in a fixed position. Distal clamp 136 is used to clamp tube 80 in a fixed position. Tube 80 is twisted by twisting clamp 136 as shown with arrow T2 to twist stent 8 and further reduce the transverse profile of stent 8.

Referring to FIG. 7L, distal tip 30′ is mounted to central tube 50 as described above.

Referring to FIG. 7M, the clamps are released and stent 8 places the portion of distal latch wire 56 where electrolytic sacrificial link R1 is situated in torsion. In other words, distal latch wire 56 prevents stent 8 from untwisting.

For stent deployment, power first is provided to sacrificial link R1. When sacrificial link R1 breaks, members 72′, 64, 80, 502, 504 and 506 rotate together about central tube 50 because they are interconnected. However, tubes 504 and 84 do not rotate. As a result, stent 8, which is mounted in seat body 64, untwists and shortens. As stent 8 shortens, tabs 60a are withdrawn from seat 64 and the distal end of the stent radially expands. Power is then provided to sacrificial link R2. When sacrificial link R2 breaks, wrap 54 loosens, proximal tabs 60b of stent 8 radially expand from under insulation portions 73 and become released from delivery guide 22.

### Delivery Guide Features for Improved Stent Loading

In a method as described above (or similar thereto), one seat rotates when loading the stent onto the delivery guide. For the rotatable type latch illustrated, relatively few assembly challenges are presented. However, in a delivery system employing wrap-style latches on both sides of the stent, greater challenges are faced. Specifically, with such a latch assembly as illustrated FIGS. 4A, 4B and 5A, the in-board section of the latch wire originates or emerges (typically) between the stent end crowns 100 and tabs 60. This way, the tabs 60 and seat fingers/extensions 66 are covered by the wrap to secure the stent for navigation, until release.

When the seat is fixed relative to the delivery guide body 58 (e.g. proximal seat 62 in FIG. 5A), the latch wire can run along the body, or pass upwards though it and be wrapped as shown. However, when the seat must rotate during loading, the latch wire advantageously rotates with the seat. In this way, it can first be wrapped about stent prior to twisting. At least, it will not have to be threaded several times past the stent—potentially subjecting insulation to damage—as the seat is rotated.

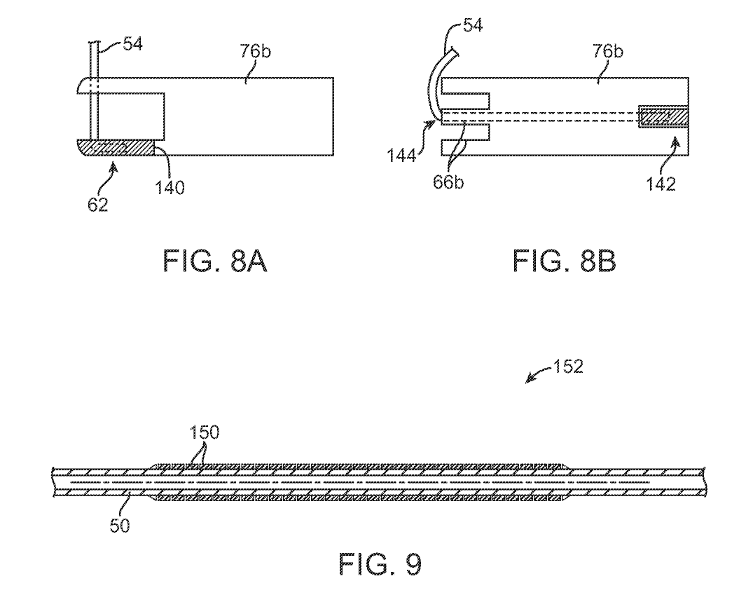

FIGS. 8A and 8B illustrate seat 62/64 and latch wire 54 assembly approaches that can advantageously used in a delivery guide system with wrap-style latches at both ends. The illustrated assemblies can serve as either near or far seats (as indicated by the numbering)—or both.

However, they are specifically adapted to allow rotation of the latch wire 54 with the seat in a loading method. When one seat is first affixed to the delivery guide as described in the method above, there is no need that it include the specific adaptations described below.

Regarding the specific embodiments, FIG. 8A shows a variation in which wire 54 is secured to a seat finger 66. Typically, an insulative polymer layer, such as a in the form of a polyimide sleeve 140 is first bonded (e.g., epoxied) to the finger. Then, the latch wire bonded to the sleeve. In the variation shown in FIG. 8B, the latch wire is instead bonded into a slot 142 created in the seat body 76. Again, an insulative polymeric layer can be interposed between the seat and latch wire 54 bonded thereto. A second glue joint 144 can also be provided at the finger end to maintain the position of the wire underlying the seat.

As indicated, the unsecured end of the latch wire in each of the illustrated variations continues for some length. This length will be sufficient to wrap over the stent to releasably secure it to the delivery guide. It is also preferably long enough to secure to the delivery guide body and pass along its length to the proximal end of the system to apply voltage to the erodable section that defines the latch region.

In a loading method, the stent is set in a complimentary position with the seat 62/64 and wire wrapped over the stent tabs/projections 66. A protective sleeve (not shown) can then be set over the wraps for clamping and twisting as shown in FIGS. 7C-7E.

FIG. 9 shows an improved support member over which the stent can be twisted according to one loading method. Here, a number of hollow cylindrical roller members 150 are set over the mandrel 50 over which the stent is twisted. As shown, the mandrel is in the form of a hypotube (which can be metallic, polymeric or a hybrid of metallic and polymeric or composite material) to allow passage of an electrical lead(s) therein.

However the mandrel is configured, the plurality of rollers are allowed to spin, roll or rotate, thereby incrementally supporting the stent as it is twisted into a compressed profile during loading. Whether due to a reduction in friction, or another factor, notable improvements in stent uniformity are observed when loaded onto an assembly 152 as shown in FIG. 9.

Still, given that profile is always at issue in producing a system with an 0.014 inch crossing profile rollers 150 can be extremely thin (e.g., having a wall thickness of about 0.0005 to about 0.0015 inches.) As such, electroformed Ni—Co pieces as described above can be advantageously employed.

Regarding the spacing, number and/or positioning of rollers 150, substantially an entire support region of the stent from its near end 12 to far end 14 contacts the cylindrical members, without underlying the stent end projections 60. At least one roller is advantageously provided for every cell/strut junction. Yet, many more (about 2-5 times as many) are desirable to achieve the incremental rotation advantages referenced above. An even higher ratio of rollers to stent length can be employed.

### Delivery Guide Features for Improved Device Navigation

FIGS. 10A-G illustrate other delivery guide body features that improve performance. In this case, the features are directed at allowing the delivery guide to track or mimic the performance of a standard high-performance guidewire despite the increased system complexity. To do so, main body of the delivery guide is suited for such use. FIG. 10A illustrates the components selected capable of such use. Unfinished body includes a superelastic NiTi hypotube 162 (roughly 165 cm) over a taper-ground stainless steel core wire 164. Power lines 166 (, which correspond to leads 52 and 52′) run under the hypotube and along core wire 164. The core wire is affixed (e.g., by soldering) to a distal superelastic NiTi “transition tube” 168 and the lines/wire received therein at a relieved or chamfered section 170 at a junction “J”. The proximal hypotube can also be connected to/soldered to the core wire. Finally, a most distal hypotube 172, upon which the stent and a far-distal atraumatic tip (neither shown) is connected. Hypotube 172 can also receive one (or more) of the power lines within its lumen. Alternatively, element 172 can comprise a solid mandrel and the one or more lines 166 run along its body (possibly protected by a polymer sleeve).

Such a system, while capable of adequate pushability and torque transmission for navigation to distal coronary anatomy or elsewhere, can include additional components. A cover in the form of a ribbon or roundwire wrap/coil 174 is added to make the outside diameter of the system substantially uniform (with the compressed stent and atraumatic tip in place) and also protect wire 166 from damage and/or secure their position. Yet, coil 174 is not intended to transmit substantial load with the system.

Coil 174 preferably includes superelastic NiTi. Such a member is conveniently wound and heat-set to size. Moreover, once one side of it is “started” on the system, the remainder can be wrapped over the underlying structures easily by spinning the device. Any excess ribbon material can be trimmed off. The starting end can first be secured by solder or epoxy, or both can be secured once the body is in place. FIGS. 10B-G provides a set of views showing the intended final product in which the transition coil 174 is provided between the most proximal and distal tubes used in the system 162 and 172, respectively.

Coil 174 (whether comprising ribbon or round wire) overlays the junction between the core wire 164 and transition hypotube 168. And because the wrap is simply rolled over the various bodies into place, it can accommodate regions having a larger diameter than the coils relaxed inside diameter (such as junction J) while snugly fitting smaller/lower regions. Such performance is not possible with a simple polymeric cover tube. Overall, the coil allows for production of a function system having a consistent outside diameter of about 0.012 to about 0.014 inches.

Referring to FIGS. 10H-I, another guide body embodiment is shown. In this embodiment, tubes 168 and 172 are combined into a single tubular member 300. Referring FIG. 10H, the distal portion of guide body 58 merges into the proximal portion of central tube 50, tube 512, and wrap 54 shown in FIG. 6J. As shown in FIG. 10H, the guide body extends proximally and includes filler “F,” which, for example, can be adhesive (e.g., epoxy) or solder, is formed into a generally cylindrical shape and encapsulates wire 54. Tube 512 terminates at about 1-2 mm from the proximal end of marker 510, which is where filler “F” begins. Filler “F” terminates where superelastic tube 174 begins and extends proximally.

Superelastic tube 174 in combination with tube 300 provides kink resistance and the desired torque transmission and in the illustrative embodiment, superelastic tube 174 is in the form of nitinol ribbon and tube 300 is in the form of tubing made from superelastic material such as nitinol tubing. However, it can be made in other forms than that shown and can be made from superelastic materials other than nitinol. Typically, central tube 50 extends into superelastic tube 174 a distance of about 10-20 mm and can be provided with a hydrophilic coating. This transition zone where central tube 50 overlaps superelastic tube 174 provides a transition between a relatively stiff region distal thereto and a relatively flexible region proximal thereto (more flexible than the relatively stiff region proximal to the transition) and provides for desirable torque transmission and pushability.

Tube 300 extends within tube 174 and also is made from superelastic material such as nitinol. Tube 300 extends proximally and has a chamfered end as shown in FIG. 10I where the tapered portion of corewire 164 is positioned and secured with solder and epoxy. Leads 52 and 52′ and corewire extend proximally to the power connection assembly as will be described in further detail below. Corewire 164 provides a path for ground and in one embodiment is high strength stainless steel (e.g., 304 or MP35). A protective sleeve (not shown) can be provided to enclose wire 52 and 52′ and the tapered portion of corewire 164 to protect the leads from the edge of the chamfered portion of tube 300. Wire 52 can be arranged to extend out from the region between tube 50 and chamfer at the proximal end of tube 300 That region is designated with reference character F in FIG. 10H and this filler can be epoxy and solder). Tube 162 extends from the proximal end of superelastic tube 174 to the power connection as well. Tube 162 also is selected to provide flexibility and pushability and in one example is nitinol with a PTFE coating. The illustrated construction is zone “C” which extends from the proximal end of the superelastic tube 174 to central tube 50 provides a relatively flexible region in the delivery guide. Zone C has a length of 15-25 cm and more typically a length of 19-22 cm. Proximally 174 is reinforced with filler (e.g., solder or adhesive) for 1-4 cm and typically 2 cm to provide additional kink resistance from zone B. And the illustrated construction in zone “B,” which has a length of 10-20 mm and in one embodiment has a length of 10 mm and extends from the proximal end of tube 174 to the beginning of the taper of corewire 164 provides a transition to relatively stiff region zone “A,” which with corewire 164 is stiffer than zone “C.” Zone A, which extends about 145-175 cm and extends in one example 155 cm, is the stiffest section and provides excellent transmission of torque and pushability to the distal end of delivery guide 22 of delivery system 20.

FIG. 10J is a sectional view of zone A taken along line A-A. FIG. 10K illustrates an alternative embodiment of zone A where leads 52 and 52′ extend within corewire 164. Zone B is less stiff than zone A, but more stiff than zone C and zone D. Zone C also is more flexible or less stiff than zone D. Zone D extends from the proximal end of central tube 50 (FIG. 10H) distally to the proximal end of marker 510 (FIG. 6J). The region of delivery guide 22 containing the stent seats and stent release mechanisms are stiffer than the stent and the portion of coil tip 30′ in zone E (FIG. 6H) is very flexible and radiopaque to provide an atraumatic lead structure for the stent and delivery guide 22. Zone E has a length of about 1 cm to about 4 cm, and more typically has a length of 2-3 cm.

A table illustrating stiffness parameters according to one embodiment of the inventions is provided below using a three point test. Generally speaking zone A, which typically has a length of about 145-165 cm is the stiffest region of delivery guide 22. Zone B is less stiff the Zone A and Zone C is more stiff than Zone E, the most floppy or flexible zone.

| | BENDING |
|---|---|
| | STIFFNESS |
| REGION OF DELIVERY GUIDE 22 | (lbf-in2) |
| Zone A | 0.0140-0.0220 |
| Composite high strength stainless steel corewire | |
| 164, with superelastic sleeve; and leads 52, 52′ | |
| Zone B | 0.007-0.013 |
| Composite tapered corewire 164 and superelastic | |
| sleeve 162 | |
| Zone C | 0.005-0.007 |
| Composite superelastic ribbon and superelastic tube | |
| and leads 51, 51′ | |
| Zone D | 0.003-0.005 |
| Composite superlative ribbon, superelastic tube and | |
| stainless steel central tube 50 followed by stainless | |
| steel central tube 50 surrounded by lead wire 52 and | |
| housing lead 52′ and housed in filler such as epoxy. | |
| Proximal Stent Restraint | 0.0025-0.0028 |
| (Cross section of FIG. 6K3) | |
| Stent | 0.0008-0.001 |
| 600 plus 602 | 0.001-0.0015 |
| (composite tube 600, tube 202, and wire 56) | |
| Zone E | 0.0001-0.0005 |
| (From the distal end of 602 to the distal end of ball | |
| 610) | |

### Power Connection

Referring to FIGS. 11A-C, diagrammatic illustrations of one embodiment of a connection portion for coupling leads 52 and 52′ to power adapter 24 and provide a ground connection is shown and generally designated with reference numeral 800. Referring to FIG. 11A, proximal connection portion 800 surrounds corewire 164. Proximal to connection portion 800 corewire 164 ends and docking extension 165 extends therefrom. Docking extension 165 provides means for extension of guide device 22. Such guidewire extensions are known in the art. Connection portion 800 comprises serially aligned gold plated tubular ground connector 802, which is soldered to stainless steel corewire 164 at 803, power connector 812 to which wire 52′ is soldered, polyimide insulation tube spacer 807, power connector 810 to which wire 52 is soldered, polyimide insulation tube spacer 808, and gold plated tubular ground connector 804, which is soldered to stainless steel corewire 164 at 805. The ground solder connections can be formed by forming an opening in each of the ground connector tubes and then providing solder in the opening to electrically connect stainless steel corewire 164 to the ground connection tubes. The power connection for power lead or wire 52′ is made by forming two holes in power connector tube 812 and lacing wire 52′ out from one opening and back through the other opening as shown in FIG. 11B. The insulation where the wire is exterior to power connector tube 812 is stripped and solder applied to electrically connect wire 52′ to power connector tube 812. The power connection for power lead or wire 52 is made in a similar manner. The power connection for wire 52 is made by forming two holes in power connector tube 810 and lacing wire 52 out from one opening and back through the other opening as shown in FIG. 11C. The insulation where the wire is exterior to power connector tube 810 is stripped and solder applied to electrically connect wire 52 to power connector tube 810. Insulative sleeve 820 is provided around corewire 164 and extends from solder connection 803 to solder connection 805 to prevent electrical shorting between 810 and 812.

Referring to FIG. 11D, one adapter configuration is shown where ground connector tube 802 is coupled to adapter ground connector 802C, power connector 812 for distal lead 52′ is coupled to adapter distal latch wire connector 852′C, power connector 810 for proximal lead 52 is coupled to adapter proximal latch wire connector 852C, and ground connector 804 is coupled to adapter ground connector 804C and the connections secured with screw fasteners. Leads for each of these connector run to connector 32 which is coupled to power supply 26.

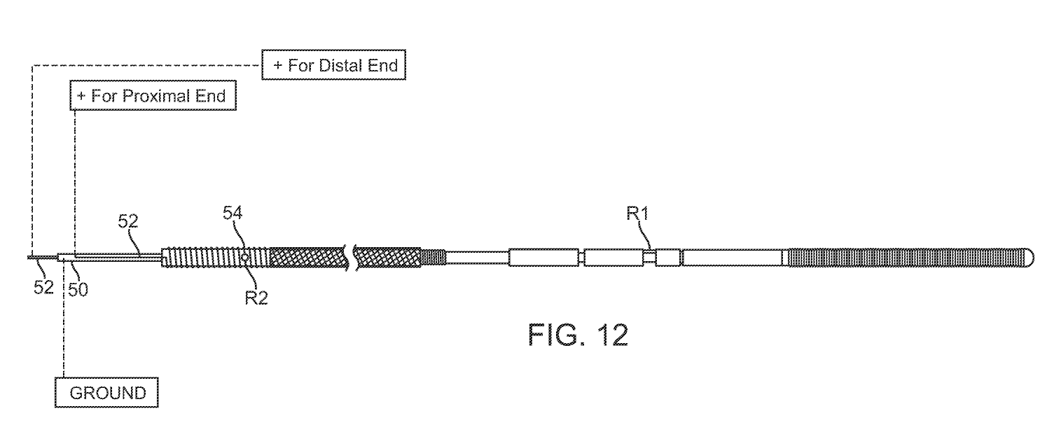

Referring to FIG. 12, a diagrammatic illustration of the circuit is shown. Power input into lead 52′ creates a circuit from sacrificial link R1 to the patient's blood and then back to ground through central tube 50 or other conductive component in delivery guide 22 and then to corewire 164. Power input into lead 52 similarly creates a circuit from sacrificial link R2 to the patient's blood and then back to ground through central tube 50 of or other conductive component in delivery guide 22 and then to corewire 164.

### Stent Tab Feature Details

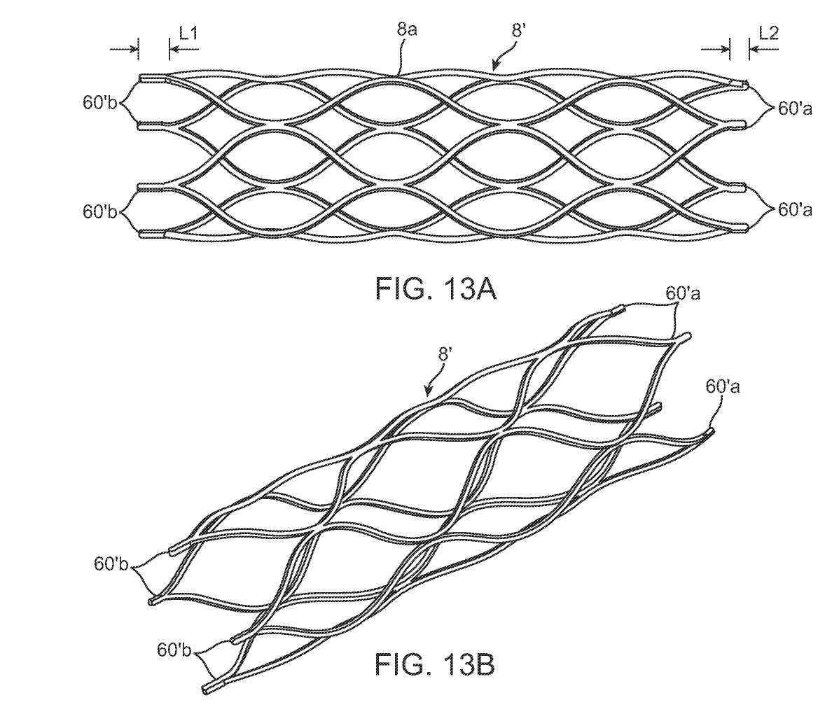

Referring to FIGS. 13A and 13B, another stent embodiment is shown and generally designated with reference numeral 8′. Stent 8′ has distal tabs 60′a and proximal tabs 60′b that extend from the closed cell stent body 8a′ and that are generally parallel to the longitudinal axis of the stent when in an unconstrained, relaxed state. In this embodiment, proximal stent tabs 60′b are longer than distal stent tabs 60′a measured in the aforementioned longitudinal axis. This configuration facilitates quick release of the distal end of the stent, while allowing the proximal end to be securely held in the event that stent relocation is desired. In one embodiment, proximal stent tabs are twice as long as stent tabs 60′a measured in the aforementioned longitudinal axis.

As referenced above, the stent also can include offset tab features offering certain advantages. The use of offset tabs as described further below offers potential for improvement not only to overall system profile, but also electrical robustness. Essentially, by lying flat, they occupy as small an envelope as possible. At the same time, the more manageable and regular profile avoids canting or turning out of plane that would put additional stress on interfacing material (such as insulative polymer layer 74). As such the tabs are less likely to cut through very thin material or force them out of position during device loading.

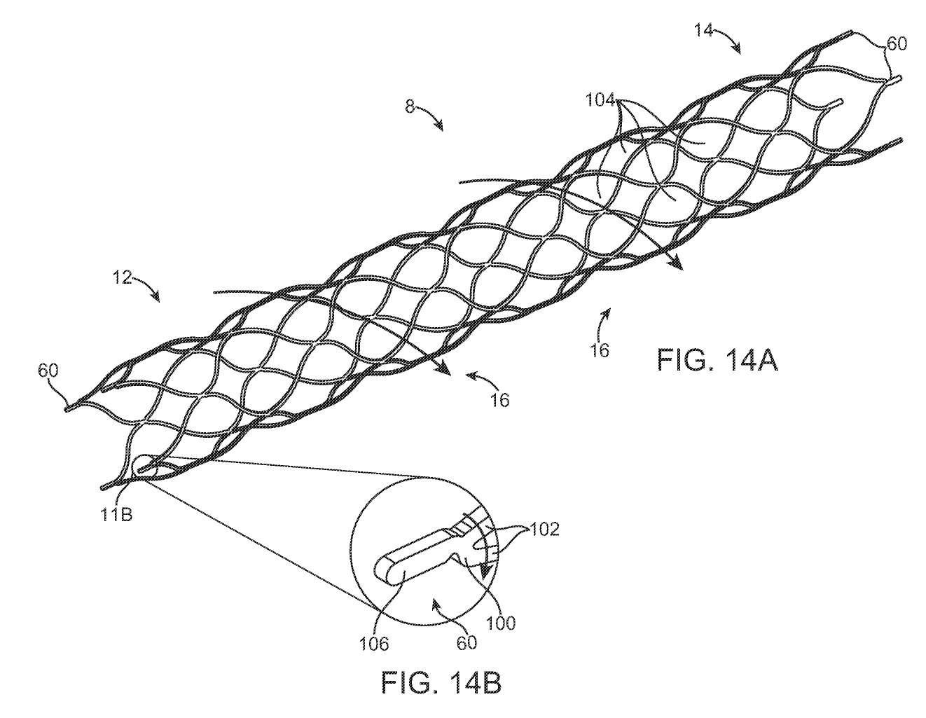

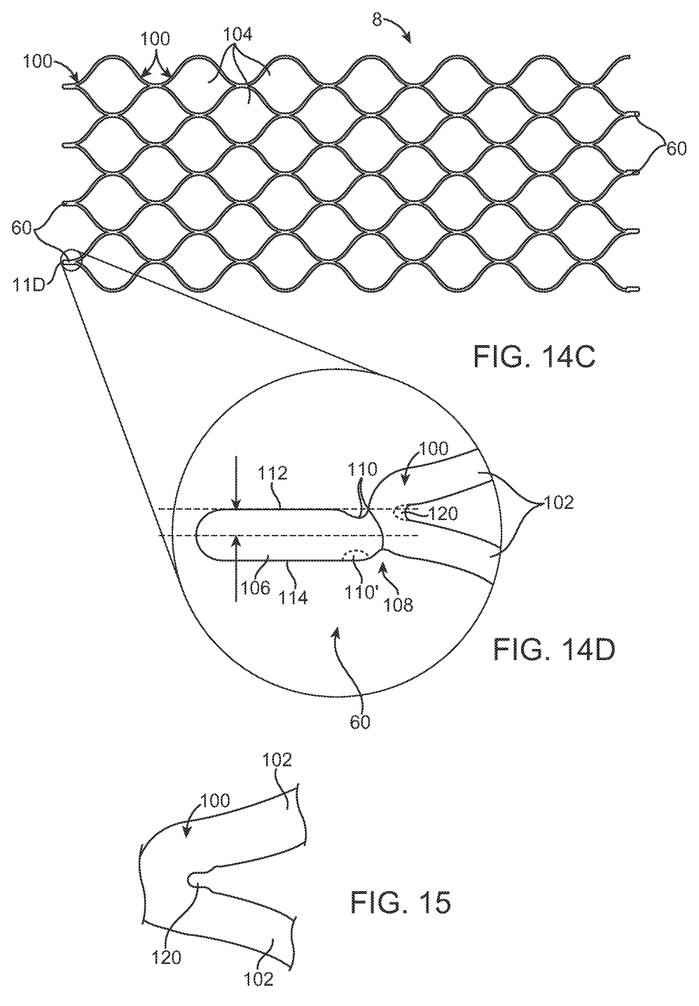

In reference to FIGS. 14A-B, stent 8 includes proximal/near medial and distal/far portions as described in connection with FIG. 2B. Given that the stent shown is symmetrical, its direction is reversible when loaded onto the delivery system.

Extensions/projections/tabs 60 comprise discrete regions to permit retention of the stent 8 on a delivery system 22. The projections illustrated are specifically adapted for retention of the stent through twisting it down into a helical reduced profile when the ends are rotated relative to one another as indicated by arrows.

The projections are connected to or extend or emanate from crown sections 100 provided between axially/horizontally adjacent struts or arms/legs 102, wherein the struts define a lattice of closed cells 104. Such closed-cell designs facilitate twist-down of the stent because the otherwise free ends of an open ended cell (or successive ring) design have a tendency to radially lift-off in a radial direction due to complex stress distributions. Whereas coil stents are twisted in bulk, their component parts are typically largely placed in tension. With the lattice-type stent designs described herein, the overall tubular body is subject to torque-based loading.

The tab configuration is adapted to address this mode of loading. Namely, with tabs axially aligned with strut cells (as shown, for example in FIGS. 6A and 6B), when the stent body is to rotation and the extensions are received within their respective seats, the tabs/extensions have a tendency to torque or “roll-over” in the direction that the adjoining crowns have been drawn. This tendency to rotate is counteracted to a degree by the member the covers the tabs/extension in the delivery guide.

However, relying on wrap wire 54 or slider 72 alone (or essentially alone) to maintain a suitable tab configuration leads to use of bulkier components. Furthermore, it does not address a tendency of off-kilter or canted tabs to bind either with complimentary seat features and/or any covering. The offset tab approach addresses each of these considerations.

As most clearly observed in FIGS. 14C-D, which offers a view of the overall pattern to which a stent can be cut, the detail section highlights an offset tab member 60. Specifically, the detail section of the figure shows tab body 106 offset from a centerline of an adjoining cell 104 defined by struts by an offset distance “O”. Body 106 is connected to crown section 100 by neck region 108. The undercut neck section serves as a virtual pivot point or living hinge allowing the tab to remain substantially straight within a corresponding seat feature while adjacent struts 102 are angled thereto when the stent is held on the delivery guide in its twisted configuration.

Undercut portions are strategically located to accommodate the bending by maintaining similar strut width around the crown. As another option, the edge-side undercut could be moved as indicated by 110′ to offer more symmetry along the tab. This option can in some instance improve tab rotation performance about the axis indicated. In other instances, it can result in strains that are too high in adjacent crown material.

More generally, the offset location of the tab(s) connection to adjoining strut crowns provide a laterally-displaced point of rotation with at least a component substantially parallel with the delivery guide around which the tabs rotate until they lie substantially flat on the delivery guide. Torque on the crown causes the bulk of a given body 106 of the tab to lie down until it contacts the underlying portion of the delivery guide body. In other words, contact between the inner edge 112 (or thereabouts) of the tabs with mandrel or hypotube 50 limits further rotation.

Typically, each end crown 100 of the stent will be capped or transitioned to an extension. In this manner, the stent can be fully constrained without portions thereof tending to lift-off the delivery guide in a pure twisting mode of diameter reduction for delivery. However, it is contemplated that the number of crowns can be reduced by taking out adjacent arm sections to turn what was a four-crown design on each end into a two-crown design. In this way, fewer projections can be used, while still providing one for every full cell at each end of the stent.

The degree of offset of the tabs as well as their width are variable. When seeking to minimize restrained stent profile, it will generally be desired that the outboard edge or extent 114 of the tabs not extend beyond (or at least not extent too far beyond) the envelope defined by adjacent crowns. Such a configuration would increase the compressed/twisted size of the implant.

The overall configuration of the projections can also vary as summarized above. Also, the direction (clockwise vs. counterclockwise relative to the stent body) that the tabs are offset can vary. Indeed, FIGS. 14B and 14D show the tabs offset in opposite directions. With a delivery guide as shown, the only significance of this selection determines which way the stent is to be twisted for loading. Yet, if the stent were to be anchored in the middle and the stent twisted in opposite directions outside this point, then the projections could be offset in opposite directions.

Still further, the projections can also vary in length, especially depending on the form of interface or mating portion it carries or forms. The projections advantageously have a length that allow for efficiently transition or transfer twisting load to the stent while occupying minimal space. Though usable with the devices, systems and methods described herein, projections longer than about one cell's length can have a tendency to wrap or twist about the delivery device body in attempted use.

For a stent and delivery system adapted to present an 0.014 crossing profile, the tabs can be approximately 0.020 inches in length and between about 0.002 and about 0.003 inches wide, thereby having a centerline offset from that of the crown/strut center by as little as about 0.001 to about 0.0025 inches and still offering substantial advantages in the loaded configuration of the stent.

In certain larger systems, the offsetting approach can offer significant benefit. However, it may not be as necessary as in smaller systems in which space is limited, and material layers are less robust. In the conditions where even 0.0005 inches thickness of material must be counted as relevant toward diameter and/or tolerance stack up, the advantages offered by the offset tabs is especially useful. Still, it should be noted that the devices, systems and methods are not limited to such.

### Stent Body Design Features

Another useful stent geometry feature is shown in FIG. 15. Here (at approximate scale relative to the enlarged section in FIG. 14B), an additional undercut section or notch 120 is taken from the crown region to allow it better flex when loaded onto the delivery guide. Notches can be employed on the tab-side crowns as well as the crowns at bridges between adjacent cells 104. The additional, highly localized flexibility is offered to improve packing of the stent already optimized for compression, given its S-curved struts.

The notch feature(s) can be used in conjunction with a stent design employing offset tabs, the reverse-engineering approach next detailed, or otherwise. Indeed, the advantages that the notch can offer can be particularly useful in instances in a stent without offset tabs or other design improvements art to be employed. Examples of such stents are presented in FIGS. 6A and 6B.

By employing a substantially parallel-wall notch (at least when first laser cut to extend the otherwise (essentially) V-shaped junction of the struts), the width of the undercut is minimized. This aspect leaves as much material as possible along the length of the struts before and after electropolishing. As such, the depth of the notched section can be finely tuned (e.g., maximized) without creating problematic strains and stresses in the stent design with the crown compresses (essentially) in-plane and/or about an axis defined by the body of the stent.

Further improvement of stent strut packing can be accomplished by wholesale redesign of the stent, as opposed to selective tuning as described above. Specifically, a method for stent design like that employed in generating the cell pattern of the stent shown in FIGS. 14A and 14C can be employed, while also accounting for the twisted component of the stent. As referenced above, the basis of the method is presented in U.S. patent application Ser. No. 11/238,646, incorporated by reference herein in its entirety. (See, e.g., FIGS. 2A-B, 5A-C, 6A-B, 7A-B, 8A-B and their associated texts, as well as paragraphs 58-64, 90-95, 101-107).

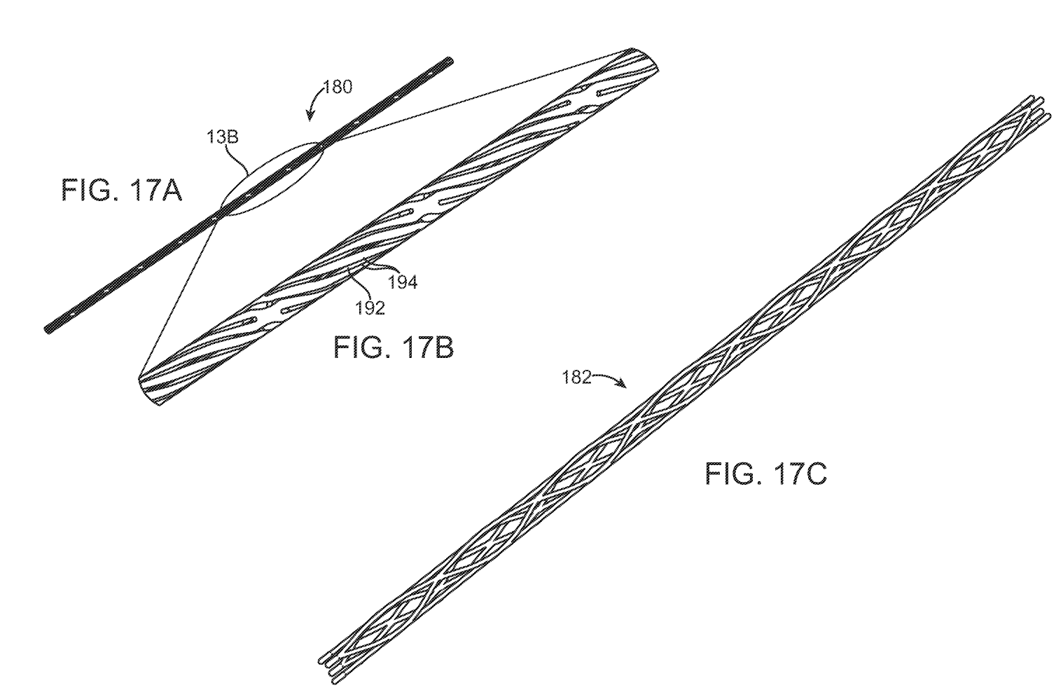

According to this method of stent design, FIGS. 17A-C show precursor stent patterns to be cut. Preferably, they are cut in tubing of the same material as the final stent to be produced. In this manner, reversibility of the process is best insured.

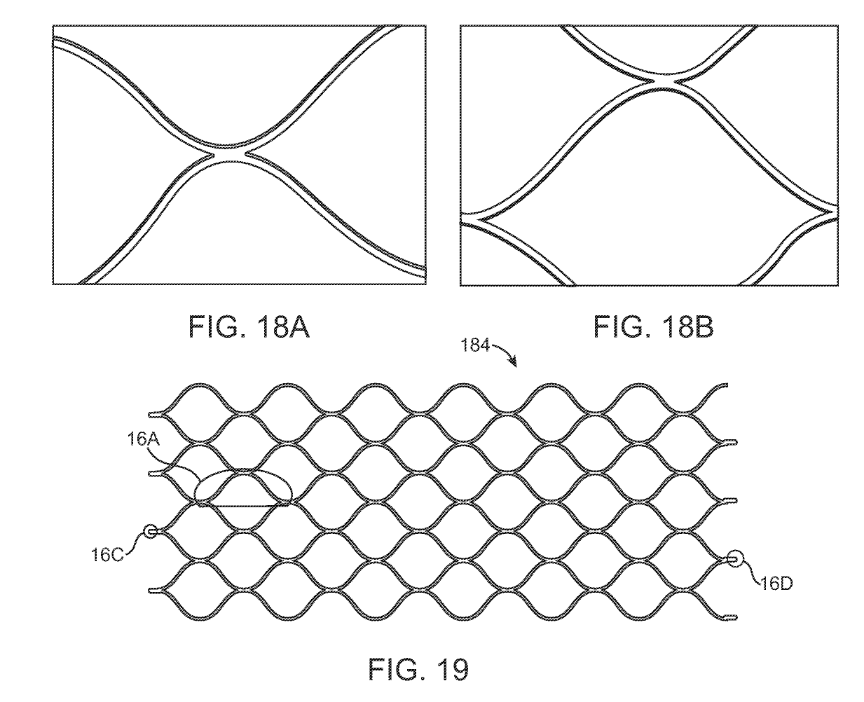

Regarding the process, a precursor stent is designed and produced having the desired compaction properties (as in FIG. 17B for a minimum diameter twisted stent 180), or nearly so (as in FIG. 17C for a small diameter twisted stent 182). With such a stent laser cut and (preferably) electropolished, it is expanded and heat-set in and expanded shape. This expansion can take one or more steps. FIGS. 18A and 18B shows the expanded pattern of a stent cut according to the FIG. 17C design.

Note, that while it may be preferred to cut the stent in its most compact form (i.e., as shown in FIG. 17B) certain challenges or limitations with manufacture can dictate doing the work at a slightly larger diameter (i.e., as shown in FIG. 17C). For the process to be useful in guiding the design of the final stent, however, the process should be performed near the intended compressed diameter of the stent. Of course, “near” is a relative term. For a stent with a 10× compression ratio, designing the precursor at less than 5× expansion can be helpful in guiding the design of a non-compacted stent by analyzing the result of expanding the precursor design. More preferably, the precursor stent is produced at 3× the compressed diameter (for a 10× design) or less, such as 2×, most preferably within about 50% of the intended fully compressed diameter of the final stent. Moreover, these values can change when designing for self-expanding stents with lower expansion ratios. Essentially, the practical limits are determined by what size ratios produce useful results in carrying out the subject method.

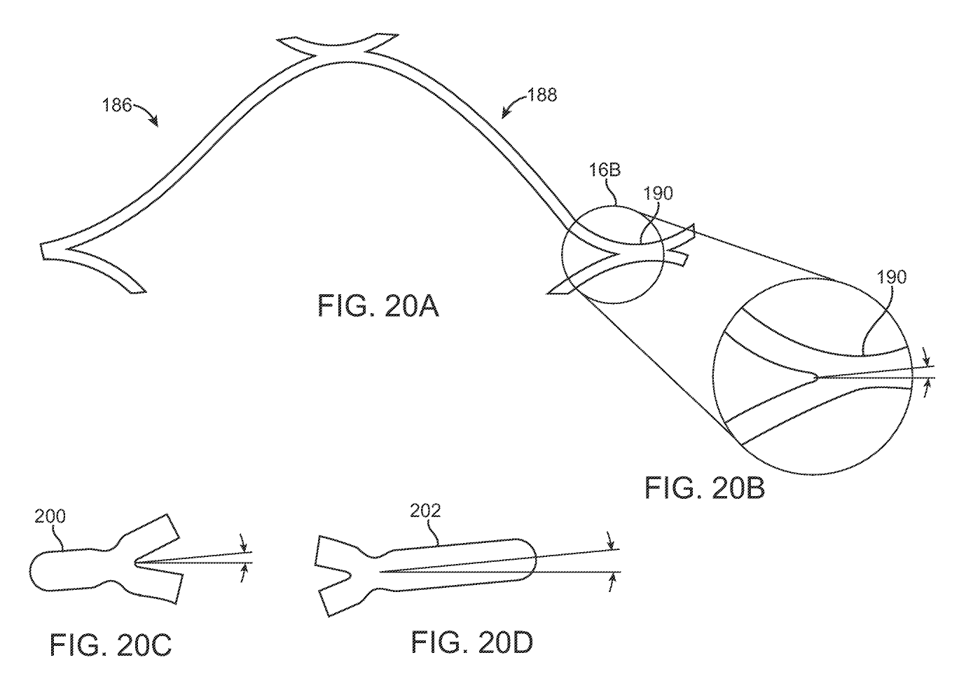

Returning to FIGS. 18A and 18B, however, features of the schematic drawings can be scaled or overlaid, especially in a CAD design of a final stent cut pattern 184 as is shown in FIG. 19. Details of pattern 184 are provided in FIG. 20A. Here different strut curve geometries 186, 188 can be observed. Also, as illustrated in FIG. 20B, which is an enlarged view of area 20B in FIG. 20A, the stent bridges 190 can be canted at an angle. All of these adaptations assist when a stent cut to the pattern in FIG. 20A is compressed and twist-loaded onto a delivery guide.

The intended result is a compressed stent that appears substantially as the inset, enlarged view in FIG. 17B, in which parallel-wall or teardrop spaces 192 are presented between adjacent struts 194 wrapping around the stent.

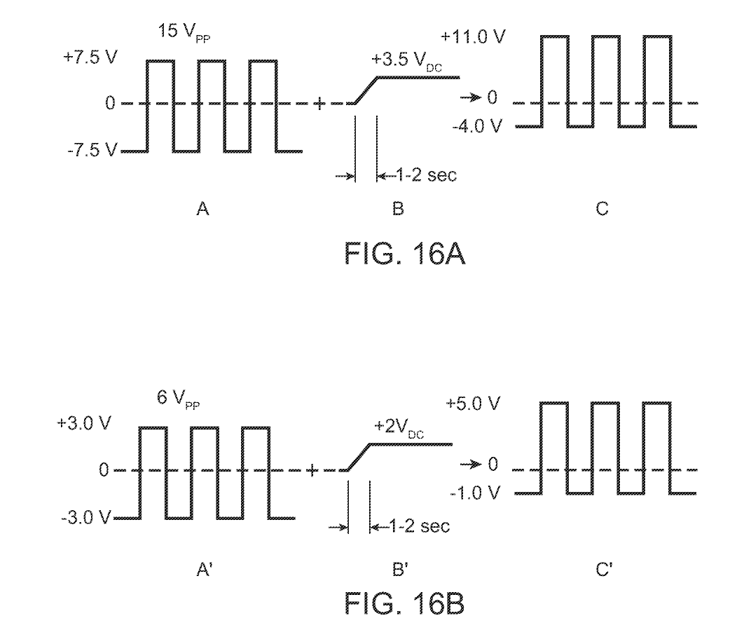

Another notable aspect of the stent design presented in FIG. 19 is the tab/extension features 200, 202. For one, the tabs are oriented at an angle relative to a major axis or lumen of the stent, as shown in FIGS. 16C-D. The angle substantially matches that of the bridge region 190. Of further note, the tabs are of different length.

This latter feature (different length tabs) is not related to improvements for compaction, but rather to facilitate stent release from the delivery guide. As shortened tab (approximately one-half the length of the other, or about 0.010 inches long) can be advantageous when employed with an un-twisting style of latch mechanism, such as distal latch mechanism 70 shown in FIGS. 4A, 4B and 5B. It can be especially useful when band 72 is fixed, because less length of the tab 200 will be needed to slide out of the seat in order to achieve (at least partial) stent release.

### Electrical Performance

In one exemplary embodiment, release of the stent is accomplished by applying a DC voltage to achieve corrosion/erosion of the implant release means. And while adding an AC voltage component for sensing purposes is known (e.g., as described U.S. Pat. Nos. 5,569,245 to Guglielmi, et al. and 5,643,254 to Scheldrup, et al.), AC voltage is preferably used herein in a very different manner.

Specifically, it has been appreciated that the use of significant AC component offset by a DC signal can dramatically improve the process of implant delivery through electrolytic corrosion. Not to be bound by a particular theory, but it is thought that efficiency gains are related to controlling blood electrocoagulation and/or having periods of higher peak voltage during the upsweep of the AC signal. The benefits derived from the AC component is especially advantageous in coronary therapy because high frequency (e.g., 10 kHz to 100 kHz or greater) AC power does not affect heart rhythm unless the waveform becomes unstable.

Controlling electro-coagulation is very important for safety reasons (e.g., in avoiding emboli formation that could lead to stroke or other complications) and also to increase the speed of corrosion. Generally speaking, while corroding a positively charged section of metal, the positive charge attracts negatively charged blood cells which coagulate on the surface of the metal. Coagulated blood cells can cover the corroding metal and slow the deployment process. Higher DC levels can be employed to push past this effect, but for safety considerations (especially in the vicinity of the heart) it is desirable to use lower DC voltages. Instead, when an AC signal is employed that drops the trough of the waveform into the negative regime, an opportunity exists to repel the negatively charged blood cells. The resulting decrease or lack of electrocoagulation offers an efficiency increase so that DC voltage can be dropped while maintaining deployment times that are subjectively acceptable to a medical practitioner (e.g., less than about 1 minute or about 30 seconds—even as little as a few seconds).

Power is preferably delivered by a custom battery-powered power supply. Most preferably, a current-control hardware and software driven (vs. software-only driven) power supply is employed. Still, various power/function generators, such as a Fluke model PM 5139 Function Generator, can be employed for experimental purposes. A square wave function is most advantageously employed in order to maximize the time spent at peak and minimum voltage levels, but sinusoidal, saw-tooth, and other variations of these forms can be employed. Still further, frequency modulated waveforms in which more or less time is spent in the positive or negative regimes can be employed.

The power profile applied to the delivery guide can be as described in U.S. patent application Ser. No. 11/265,999, incorporated herein by reference in its entirety. Specifically, a square wave at about 100 kHz with a 10V peak to peak (10Vpp) AC component that is offset by a 2.2V DC signal can be employed. The superposition of signals results in a square wave with a 7.2V peak and −3.8V trough. With the addition of an AC profile of at least 4Vpp, however, the DC component could drop to as low as about 1V to about 1.5V giving a resulting waveform with a peak from 3 to 3.5V and a trough from −1 to −0.5V and still offer an acceptable rate of corrosion. More typically, a square wave at about 100 kHz with a 20V peak to peak (20Vpp) AC component that is offset by a maximum of 9.0VDC signal can be employed. The superposition of signals results in a square wave with a maximum of 19V peak and −1.0 trough.

In porcine blood, it was determined that a peak waveform voltage of above 8V begins to cause electrocoagulation, even with trough voltages of −6 to −7V. The level of electrocoagulation varies with the level of the DC component and the size of the piece of metal to be eroded, but usually the peak voltage at the site of the latch(es) should remain below 9V and most often below 8V to avoid appreciable electrocoagulation.

In view of the above, and further for safety reasons—especially in the vicinity of the heart—it may be desirable to maintain the DC component of the power applied at the latch(es) between about 1 and about 5V, and more preferably between about 1.75 and about 3V, and possibly most preferably between about 2 and about 3V. The AC waveform employed will generally then be selected to generate a peak at the point of action below about 9V and usually below about 8V, with 7 to 7.5V being typical per the above. Accordingly, the resultant power profile applied at the point of corrosion can have a peak or maximum between about 4 and about 9V, and a minimum of about −0.5 to about −5V. Within this range (and in certain circumstances, outside the range, given situations where some amount of electrocoagulation is acceptable), more effective combinations exist as detailed herein and as can be apparent to those with skill in the art in review of the present disclosure.

A highly effective power profile is shown in FIG. 16A. This figure illustrates the combination of AC component “A” with DC component “B” to yield the power profile “C” applied to the delivery guide. Due to impedance of the system (in this case, modeled at as a stainless steel wire of 6 to 6.5 ft at 0.0012 inch diameter having an impedance of about 2-3 kΩ) a significant drop in the AC voltage is expected, with some drop in the DC voltage as well. As such, the latch(es) on the delivery guide can “see” or are subject to a power profile more like that shown in FIG. 16B in which components A′ and B′ are combined to yield overall power profile C′.

Per the theoretical system shown in FIGS. 16A and 16B, then, power is applied at 15 Vpp at 100 kHz with a DC offset of 3.5 V; the power delivered (to the latch wire(s) is approximately 6 Vpp with a DC offset of about 2 V. The actual power delivered will vary with details of device construction, material selection, etc.

Irrespective of such variability, an important aspect of the power profile (both as applied and delivered to the erodable material) concerns the manner of its control. Another important aspect concerns the DC component application.

As for the former consideration, as noted above, a current-control power supply is advantageously employed. In a current-controlled implementation, the DC voltage can be allowed to “float” upwards to a maximum of 9.5 V. The AC component remains constant and often yields a net signal in the blood-repulsive regime, but the system can continue to deliver current to produce highly consistent latch erosion performance.

Also, in a current-controlled implementation, current can be monitored with precision and offers ease of implementation in a custom system as compared to voltage control hardware. Further, the reaction time of the system can be controlled such that any spike in current persists only for about 1/100,000 of a section. In the kHz range, heart tissue will not respond to any such anomaly. Certain hardware implementations can be preferable over other software implementations where current reaction times can be expected in about 1/200 of a second, or the 50 Hz range—a particularly vulnerable regime for electrical/myocardial interaction. However the control system is implemented, frequencies to which the heart is susceptible should be avoided.

As for DC component application, references to components B and B′ in FIGS. 17A and 17B illustrate an advantageous approach. Specifically, DC voltage (hence, power) is increased gradually. By doing so (e.g., over a period of time of about 1 to about 2 seconds), a step function that the heart can react to is avoided. In practice, a shorter ramp-up time can be acceptable (e.g., on the order of 0.10 to about 0.25 or about 0.5 seconds) and longer time frames can be employed (e.g., as much as 5 or 10 seconds).

A ramp-up as shown and described offers additional safety to the system as observed in numerous animal trials. Further, the short delay of 1-2 seconds in reaching full power to drive the electrolytic erosion of the latch members is not significantly inconvenient in terms of waiting for system action. Indeed, with a power profile as shown in FIG. 17A, latch erosion times (with a proximal latch comprising 0.0078 diameter stainless steel and a distal latch wire comprising 0.0012 stainless steel wire with approximately 0.002 to about 0.005 inches exposed and the remainder insulated) averages only 3 to 15 seconds. It is also noted, that while the wire can be thicker in a rotatable latch assembly than a wrap-style assembly, the rotatable assembly release times can be the lower of the two due to the load on the latch wire exerted by the stent.

Last, it is noted that in instances when release may not occur as desired, as determined by monitoring by control hardware/software, that a “ramp-down” regimen analogous to the “ramp-up” aspect of the power profile can be desirable. Such a feature can be desirable in order to add a further measure of safety to account from device mishandling, etc.

The following table sets forth example power parameters for a stent having a construction as shown in FIG. 6B or 6E and having a compressed delivery outer diameter of about 0.014 inch.

Value

| | | |
|---|---|---|
| | R1 | R2 |
| Parameter | (distal) | (proximal) |

| | |
|---|---|
| AC voltage | 5-20 Vₚₚ (13-nomimal) |
| AC duty cycle | 50% |
| AC frequency | 110 kHz |
| AC ramp up and ramp down time | 0.3 sec |
| DC Voltage limit | 9.0 V |
| DC current output for loads between | 200 μA |
| 5 kΩ and 45 kΩ | |
| DC ramp up and ramp down time | 0.1-5 sec |
| AC wave form | Square |

### Variations

Also contemplated herein are methods that can be performed using the subject devices or by other means. The methods can all comprise the act of providing a suitable device. Such provision can be performed by the end user. In other words, the “providing” (e.g., a delivery system) merely requires the end user obtain, access, approach, position, set-up, activate, power-up or otherwise act to provide the requisite device in the subject method. Methods recited herein can be carried out in any order of the recited events which is logically possible, as well as in the recited order of events.

Exemplary embodiments, together with details regarding material selection and manufacture have been set forth above. As for other details of the presently described subject matter, these can be appreciated in connection with the above-referenced patents and publications as well as generally know or appreciated by those with skill in the art. For example, one with skill in the art will appreciate that a lubricious coating (e.g., hydrophilic polymers such as polyvinylpyrrolidone-based compositions, fluoropolymers such as tetrafluoroethylene, hydrophilic gel or silicones) can be placed on the core member of the device, if desired to facilitate low friction manipulation. The same can hold true with respect to method-based aspects in terms of additional acts as commonly or logically employed.

Further, any feature described in any one embodiment described herein can be combined with any other feature of any of the other embodiments whether preferred or not.

In addition, though the devices, systems and methods described herein have been presented herein in reference to exemplary embodiments, optionally incorporating various features, the devices, systems and methods described herein are not to be limited to that which is described or indicated as contemplated with respect to each variation. Various changes can be made to the subject matter described herein, and equivalents (whether recited herein or not included for the sake of some brevity) can be substituted without departing from the true spirit and scope of the disclosure. In addition, where a range of values is provided, it is understood that every intervening value, between the upper and lower limit of that range and any other stated or intervening value in that stated range is encompassed within the disclosure. Furthermore, where discrete values or ranges of values are set forth, it should be noted that the devices, systems and methods described herein are not limited to such.

Also, it is contemplated that any optional feature of the inventive variations described can be set forth and claimed independently, or in combination with any one or more of the features described herein. Stated otherwise, it is to be understood that each of the improvements described herein independently offer a valuable contributions to the state of the art. So too do the various other possible combination of the improvements/features described herein and/or incorporated by reference, any of which can be claimed.

1. A stent delivery system including an elongate delivery guide comprising: a stent comprising a near end, a far end and a structure extending therebetween, the stent further comprising a near and a far mating portion at the near and far ends of the stent, near and far seats at a far portion of the delivery guide, a mating portion being received in each seat, at least one helical wrap including an electrolytically erodable section, the wrap at least partially covering at least one of the seats and mating portions received therein, and an insulative polymer sleeve interposed between the wrap and the mating portions.

2. The system of claim 1, wherein the stent is in a twisted configuration.

3. The system of claim 1, wherein only one seat is covered by a helical wrap, and wherein at least one seat is rotatable upon release of an electrolytically erodible member.

4. The system of claim 1, wherein the sleeve has a thickness of about 0.001 inches or less.

5. The system of claim 1, wherein the sleeve is slit along a plurality of lines between the mating portions.

6. A stent delivery system including an elongate delivery guide comprising: a stent comprising a near end, a far end and a structure extending therebetween, the stent further comprising a near and a far mating portion at the near and far ends of the stent, near and far seats at a far portion of the delivery guide, one mating portion being received in one seat and the other mating portion being receiving in the other seat, one of the seats being rotatable, and near far restraints for holding portions of the stent in a compressed state, one of the restraints including a helical wrap having an electrolytically erodable section, the wrap at least partially covering one of the seats and mating portions received therein.

7. The system of claim 6, wherein the stent is in a twisted state.

8. The system of claim 7, wherein the stent has a closed cell construction.

9. The system of claim 6, further including a sleeve positioned around one of the seats and the wrap is secured to the seat over the sleeve.

10. A method of loading a stent delivery system comprising: securing the first end of a stent having first and second ends to a first seat that is fixed to a delivery guide, the first end being secured to the first seat with a wrapping member, securing the second end of the stent to a second seat that is coupled to the delivery guide, twisting the stent into a twisted configuration while the second end of the stent is secured to a second seat with a restraint, and fixing the second seat to the delivery guide.

11. The method of claim 10, wherein the second restraint is fixed to the delivery guide after twisting the stent.

12. A method of implant delivery comprising: introducing an implant delivery system in an electrolytic fluid; and applying electrical power to a delivery guide having at least one electrolytically erodable member, the power having an AC voltage component with a peak-to-peak configuration of at least about 5V, and a DC voltage signal of at least about 1V, wherein the DC component is increased from zero to a maximum over a period of at least about 0.1 seconds.

13. The method of claim 12, wherein the DC component is increased from zero to a maximum over a period of at least about 0.5 seconds.

14. The method of claim 12, wherein the DC component is increased from zero to a maximum over at least about 1 second.

15. The method of claim 12, wherein the DC voltage varies to deliver a constant current during electrolytic erosion.

16. The method of claim 15, wherein the DC voltage varies between about 1V and 9.5V.

17. The method of claim 12, wherein the AC voltage component has a peak-to-peak configuration of 20V or less.

18. The method of claim 12, wherein the AC voltage component has a peak-to-peak configuration of 15V or less.

19. The method of claim 12, wherein the AC component has a substantially square-wave profile.

20. The method of claim 12, where the power applied includes a negative voltage signal.

21. The method of claim 20, wherein the power-applied always includes a negative voltage signal.

22. The method of claim 12, wherein the power delivered to each electrically erodable member having an AC voltage component has a peak-to-peak configuration of at least about 5V, and a DC voltage signal of at least about 1V.

23. A implant delivery guide body comprising: an elongate body, the body comprising a proximal metal tube, a distal metal tube, a corewire, and a superelastic helical wrap, the core wire connecting the proximal and distal tubes, the wrap overlaying at least one junction between the proximal and distal tubes.

24. The delivery guide body of claim 23, wherein the wrap comprises NiTi material.

25. The delivery guide body of claim 23, wherein the covered junction comprises a joint.

26. The delivery guide of body claim 25, wherein the joint comprises conductive material.

27. The delivery guide of body claim 23, wherein the wrap is soldered at two ends.

28. The delivery guide of body claim 23, wherein the wrap comprises ribbon.

29. The delivery guide of body claim 23, wherein the wrap comprises round wire.

30. The delivery guide of body claim 23, wherein the delivery guide has a substantially uniform outside diameter due to the wrap.

31. The delivery guide of body claim 30, wherein the outside diameter ranges from about 0.012 to about 0.014 inches.

32. A stent delivery system comprising: an implant delivery guide body comprising a proximal metal tube, a distal metal tube, a corewire, and a superelastic helical wrap, the core wire connecting the proximal and distal tubes, the wrap overlaying at least one junction between the proximal and distal tubes, and a stent releasably mounted adjacent a distal end of the guide body.

33. A stent delivery system comprising: an elongate-delivery guide body, and a stent releasably secured to the delivery guide body, the stent held in a twisted, compressed profile for delivery, over a mandrel, a plurality of hollow cylindrical members interposed between the stent and the mandrel, wherein the hollow members are rotatable about the mandrel at least prior to holding the stent in its delivery profile.

34. The stent delivery system of claim 33, wherein the mandrel is formed of metal tubing.

35. The stent delivery system of claim 34, wherein the cylindrical elements have a wall thickness of between about 0.0005 and about 0.0015 inches.

36. The stent delivery system of claim 33, wherein substantially an entire support region of the stent contacts the cylindrical members.

37. The system of claim 36, wherein the stent includes end projections, and no cylindrical members are in contact with the end projections.

38. The system of claim 33, wherein the plurality of hollow cylindric members, comprises at least 3 members.

39. A method of loading a stent delivery system, the method comprising: rotating at least one of a stent not over a mandrel, onto a plurality of rollers on the mandrel; progressively spinning the rollers as the stent progressively assumes a compressed diameter, and securing the stent to the delivery system.

40. The method of claim 39, wherein the stent is a non-coil type stent.

41. The method of claim 39, wherein a first end of the stent is initially secured to the delivery system, and a second end is rotated and then secured of the delivery system.

42. A self-expanding stent comprising: a body portion have a closed cell lattice construction, a longitudinal axis when in relaxed state, and distal and proximal ends; a plurality of distal projections extending from said distal end and a plurality of proximal projections extending from said proximal end, the distal and proximal projections extending in a direction generally parallel to the longitudinal axis, and, the proximal projections being longer than said distal projections.

43. The self-expanding stent of claim 42 wherein said proximal projections are at least twice as long as the distal projections.

44. A stent delivery guide comprising a delivery guide having a first length having a proximal and distal end portion, a second length having a proximal and distal end portion, a self-expanding stent having a proximal and distal end portion, and a coil having a proximal and distal end the first length distal end portion being coupled to the second length proximal end portion, the second length distal end portion being coupled toe the proximal end of the stent and the distal end of the stent being coupled to the proximal end of the coil, which forms the distal tip of the delivery guide, the first length being less flexible than the second length.

45. The stent delivery guide of claim 44 where the stent is in a twisted state and has a closed cell construction.

46. The method of claim 10 wherein the restraint comprises, a coil.

47. A method for delivering a balloon catheter to a lesion site comprising:
positioning a stent carrying delivery guide with a self-expanding stent releasably coupled thereto at a location in a vessel with the stent near a lesion site;
tracking a balloon catheter with an expandable balloon over the stent carrying delivery guide to the lesion site and dilating the lesion site with the balloon catheter;
moving the balloon catheter to allow release of the stent at the lesion site, while maintaining the balloon catheter tracked over the stent carrying delivery guide;
deploying the stent from the stent carrying delivery guide at the lesion site;
moving the balloon catheter over the delivery guide and positioning the balloon catheter balloon at the lesion site; and
manipulating the balloon catheter to effect post stent deployment dilation of the lesion site.
